(19) United States
(12) Patent Application Publication
(10) Pub. No.: US 2026/0294444 A1
(43) Pub. Date: Oct. 1, 2026

(54) WEDGE OSTEOTOMY BLADE

(71) Applicant: Treace Medical Concepts, Inc., Ponte Vedra, FL (US)

(72) Inventors: Sean F. Scanlan, Jacksonville, FL (US); Jared White, St. Augustine, FL (US); Amber Shane, Orlando, FL (US)

(21) Appl. No.: 19/578,985

(22) Filed: Mar. 26, 2026

Related U.S. Application Data
(60) Provisional application No. 63/778,067, filed on Mar. 26, 2025.

(51) Int. Cl. A61B 17/14

(52) U.S. Cl. CPC A61B 17/14

(57) ABSTRACT

An apparatus, system, and method are disclosed for performing wedge osteotomies. In some implementations, a wedge osteotomy blade includes a body having a cutting edge with a plurality of teeth exhibiting nonuniform thickness and progressively increasing dimensions across its width. The blade is configured to form precise wedge-shaped bone resections in a single surgical pass. The blade's teeth may feature varying rake angles and strategically positioned relief areas to enhance cutting efficiency, minimize vibration, and optimize bone debris evacuation. Certain embodiments incorporate asymmetrical tooth profiles to further enhance directional control and precision during cutting. Additional embodiments include teeth configured with differential engagement angles, providing controlled aggressiveness and efficiency during bone resection. The apparatus is designed for compatibility with standard oscillation handpieces, facilitating simplified surgical workflow. A method of use includes a dorsal resection approach to form controlled, wedge-shaped resections efficiently and predictably.

### CROSS REFERENCE TO RELATED APPLICATIONS

This application claims priority to and the benefit of U.S. Provisional Patent Application No. 63/778,067, filed Mar. 26, 2025, the entire contents of which is hereby incorporated by reference.

### TECHNICAL FIELD

The present disclosure relates to surgical devices, systems, instruments, and methods. More specifically, the present disclosure relates to bone cutting tools for use with powered oscillation handpieces and/or methods of designing and using the same.

### BACKGROUND

Various bone conditions may be corrected using surgical procedures, in which one or more tendons, ligaments, and/or bones may be cut, replaced, repositioned, reoriented, reattached, reduced, fixated and/or fused. These surgical procedures require the surgeon to accurately form one or more osteotomy cuts.

A variety of saw blade designs are used in orthopedic procedures, depending on the anatomy involved, the saw blade manufacturer, and clinician preference. Typically, a surgical saw blade is mounted on a powered oscillation handpiece, which the clinician grasps and activates to induce motion in the blade, allowing its teeth to cut into bone. In procedures involving small bones, such as those in the hand or foot, making precise osteotomy cuts in a single pass is especially important. Multiple passes increase the risk of excess bone removal or misalignment, which can affect surgical outcomes due to the limited size and tolerance of small bone structures.

In procedures involving small bones, such as those in the hand or foot, making precise osteotomy cuts in a single pass is particularly important. Multiple passes increase the risk of excess bone removal, cut misalignment, and inconsistent wedge geometry, all of which can negatively impact the outcome. This is especially true for wedge osteotomies, where accuracy in angle, depth, and position is important to achieving the desired correction. Forming a wedge through repeated passes can lead to asymmetry or loss of control over the resection plane. Accordingly, a need exists for a blade configured to create a wedge osteotomy in a single pass can reduce these risks by preserving bone stock, improving alignment, and streamlining the surgical workflow.

### SUMMARY

The various apparatus, devices, systems, and methods of the present disclosure have been developed in response to the present state of the art, particularly addressing problems and unmet needs in performing wedge osteotomies.

The present disclosure relates to wedge osteotomy blades configured for cutting bone, particularly in surgical applications involving powered oscillation handpieces. These blades incorporate features that facilitate controlled and precise bone resections while maintaining compatibility with conventional oscillating surgical instruments.

In one embodiment, an apparatus includes a body, an oscillation mount, and a cutting edge. The body may extend along a longitudinal axis. The body includes a proximal end and a distal end. The body can include a cutting edge at a distal end.

The oscillation mount is positioned at the proximal end, the oscillation mount is configured to couple the apparatus to a powered oscillation handpiece.

The cutting edge may be positioned at the distal end of the body, the cutting edge may include a plurality of cutting teeth. In certain embodiments, the plurality of cutting teeth have a nonuniform thickness across the width of the cutting edge. In one embodiment, the nonuniform thickness is a dimension perpendicular to a height and a width of each tooth. In one embodiment, the thickness of the plurality of cutting teeth progressively increases from one end of the cutting edge to an opposite end of the cutting edge.

Each tooth of the plurality of cutting teeth may include a base having a first side and a second side; a tip; a height measured from the base to the tip; a width measured form the first side of the base to the second side of the base; a superior side that extends from the base to the tip; and an inferior side that extends from the base to the tip. In certain embodiments, the first side has a first face having a first rake angle and the second side has a second face having a second rake angle. In one embodiment, one of the plurality of teeth includes a center tooth positioned midway between a first side of the cutting edge and a second side of the cutting edge and the first rake angle is substantially the same as the second rake angle. Furthermore, in one embodiment, a first plurality of teeth between the first side of the cutting edge and the center tooth have rake angles that bias the tips of the first plurality of teeth away from the center tooth; and a second plurality of teeth between the second side of the cutting edge and the center tooth have rake angles that bias the tips of the second plurality of teeth away from the center tooth.

In one embodiment, the cutting edge includes a single row of cutting teeth on a first side and at least two rows of teeth on a second side. In another embodiment, the cutting edge includes at least one channel that intersects cutting teeth of the cutting edge. The cutting edge may define an arc at the distal end of the apparatus such that a subset of the plurality of cutting teeth engage with a target material during cutting.

In another embodiment, the disclosure provides a wedge osteotomy blade for forming a wedge osteotomy. The wedge osteotomy blade may include a body having a proximal end, a distal end, a superior side, an inferior side, a left side, a right side, a proximal side that includes the proximal end, a distal side that includes the distal end, and a longitudinal axis that extends between the proximal end and the distal end, a lateral axis that extends between the left side and the right side, and a vertical axis that extends between the superior side and the inferior side; an oscillation mount at the proximal end, the oscillation mount configured to couple the wedge osteotomy blade to a powered oscillation handpiece; and a plurality of cutting teeth at the distal end of the body. The at least one cutting tooth includes a bone cutting feature, the bone cutting feature includes an engagement angle measured relative to the lateral axis and a length measured relative to the vertical axis; and the bone cutting feature of a first cutting tooth is longer than the bone cutting feature of a second cutting tooth, the first cutting tooth being positioned closer to the right side than the second cutting tooth.

In certain other embodiments, the bone cutting feature may include an edge, and an engagement angle may be perpendicular to the lateral axis. Alternatively, or in addition, the bone cutting feature includes at least two edges of a single cutting tooth, the single cutting tooth includes a channel in the tip of the single cutting tooth. Alternatively, or in addition, the bone cutting feature includes an edge of two separate cutting teeth, the two separate cutting teeth separated by a channel. In one embodiment, the bone cutting feature includes a first engagement angle and a second engagement angle, the first and second engagement angles summing to 180 degrees. In another embodiment, two or more cutting teeth of the plurality of cutting teeth includes a bone cutting feature and the engagement angles of the two or more bone cutting features are substantially the same. In another embodiment, two or more cutting teeth of the plurality of cutting teeth include or form a bone cutting feature and the engagement angles of the two or more bone cutting features are different. In one embodiment, one or more of the plurality of cutting teeth define a superior cut line; one or more of the plurality of cutting teeth define an inferior cut line; and the superior cut line and the inferior cut line converge at a vertex having a wedge angle. The wedge angle may range from between about 3 degrees to about 10 degrees. In one embodiment, the wedge angle is 6 degrees. Certain embodiments, of a wedge osteotomy blade may include at least one relief between the plurality of cutting teeth and the body.

The present disclosure may include a method for performing a wedge osteotomy. The method includes coupling a powered oscillation handpiece to a wedge osteotomy blade, the wedge osteotomy blade including: a body having a proximal end, a distal end; an oscillation mount at the proximal end, the oscillation mount configured to couple the wedge osteotomy blade to the powered oscillation handpiece; a cutting edge at the distal end of the body, the cutting edge comprising a plurality of cutting teeth that define a cut face; and where a thickness of the cut face defined by the plurality of cutting teeth increases laterally across the cutting edge.

The method further includes activating the powered oscillation handpiece such that the wedge osteotomy blade oscillates; positioning the cut face such that a thinnest part of the cut face substantially aligns with an apex for a wedge osteotomy, the wedge osteotomy comprising two planes that connect to the apex and extend to at least one bone surface opposite the apex, the at least one bone surface comprising cortical bone of one or more bones; and moving the oscillating wedge osteotomy blade into the one or more bones until the oscillating wedge osteotomy blade exits the one or more bones on a side of the one or more bones opposite of where the oscillating wedge osteotomy blade enters the one or more bones.

The method may also include the aspect where the cut face includes at least one thicker region and at least one thinner region and wherein moving the oscillating wedge osteotomy blade into the one or more bones forms the wedge osteotomy in a single pass, wherein the progressively increasing thickness of the resection results from the thicker region creating a greater separation between the two planes of the wedge osteotomy, and the thinner region creating a smaller separation between the two planes of the wedge osteotomy.

These embodiments provide wedge osteotomy blade for forming a wedge osteotomy in an efficient and effective manner.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1 is a perspective view of an apparatus according to one embodiment.

FIG. 2A is a perspective dorsal view of bones of a foot.

FIG. 2B is a perspective lateral view of bones of a foot.

FIG. 2C is a perspective medial view of bones of a foot.

FIG. 2D is a perspective dorsal view of bones of a foot.

FIG. 2E is a view of a foot illustrating common planes of reference for a human foot.

FIG. 3A is a plan view of a superior side of a wedge osteotomy blade according to one embodiment.

FIG. 3B is a plan view of an inferior side of a wedge osteotomy blade according to one embodiment.

FIG. 3C is a distal side view of a wedge osteotomy blade according to one embodiment.

FIG. 3D is a proximal side view of a wedge osteotomy blade according to one embodiment.

FIG. 3E is a right-side view of a wedge osteotomy blade according to one embodiment.

FIG. 3F is a left-side view of a wedge osteotomy blade according to one embodiment.

FIG. 4 is a schematic drawing illustrating a rake angle.

FIG. 5A is closeup view of teeth of a wedge osteotomy blade according to one embodiment.

FIG. 5B is a plan view of a distal end of a wedge osteotomy blade according to one embodiment.

FIG. 6A is closeup view of teeth of a wedge osteotomy blade according to one embodiment.

FIG. 6B is perspective view of a wedge osteotomy blade according to one embodiment.

FIGS. 7A-7F illustrate different patterns for bone cutting features according to one embodiment.

FIG. 8 is a distal side view of a wedge osteotomy blade according to one embodiment.

FIG. 9A is a distal side view of a wedge osteotomy blade according to one embodiment.

FIG. 9B is a distal side view of a wedge osteotomy blade according to one embodiment.

FIG. 9C is a distal side view of a wedge osteotomy blade according to one embodiment.

FIG. 10 is a flowchart diagram depicting a method for performing a wedge osteotomy, according to one embodiment.

FIGS. 11A, 11B, and 11C illustrate stages in a surgical procedure using a wedge osteotomy blade according to one embodiment.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the disclosure but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region.

“Bone surface” refers to a surface of a bone.

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g., PLLA) and/or others. In one embodiment, a body may include a housing or frame, or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “end” refers to a part or structure of an area or span that lies at the boundary or edge. An end can also refer to a point that marks the extent of something and/or a point where something ceases to exist. An end can also refer to an extreme or last part lengthwise of a structure or surface. (search “end” on Merriam-Webster. com. Merriam-Webster, 2021. Web. 4 Aug. 2021. Modified.)

“Longitudinal axis” or “Long axis” refers to an axis of a structure, device, object, apparatus, or part thereof that extends from one end of a longest dimension to an opposite end. Typically, a longitudinal axis passes through a center of the structure, device, object, apparatus, or part thereof along the longitudinal axis. The center point used for the longitudinal axis may be a geometric center point and/or a mass center point.

“Lateral axis” refers to an axis of a structure, device, object, apparatus, or part thereof that extends horizontally or transversely, typically perpendicular or substantially perpendicular to the longitudinal axis. Generally, a lateral axis passes through a central region of the structure, device, object, apparatus, or part thereof, and extends between opposing lateral sides. The reference center point used to define or characterize the lateral axis may include, without limitation, a geometric center point, a mass center point, or another suitable reference feature or measurement relevant to the structure, object, or device. (Defined in conjunction with ChatGPT 4.5 Version, Mar. 18, 2025).

“Vertical axis” refers to an axis of a structure, device, object, apparatus, or part thereof that extends vertically or substantially vertically, typically perpendicular or substantially perpendicular to both the longitudinal axis and lateral axis. Typically, a vertical axis passes through a central region of the structure, device, object, apparatus, or part thereof, extending between superior (upper) and inferior (lower) surfaces or ends. The reference center point used to define or characterize the vertical axis may include, without limitation, a geometric center point, a mass center point, or another suitable reference feature or measurement relevant to the structure, object, or device. (Defined in conjunction with ChatGPT 4.5 Version, Mar. 18, 2025).

“Trajectory” refers to a path a body travels or a path configured for a body to travel through space. (Search “trajectory” on wordhippo. com. WordHippo, 2023. Web. Modified. Accessed 13 Jun. 2023.)

“Oscillation mount” refers to a mount device, apparatus, component, module, part, piece, mounting structure, or the like configured, designed, and/or engineered to facilitate the attachment or coupling of a tool, blade, apparatus, device, or component to an oscillation handpiece (powered or unpowered). The oscillation mount may include features for secure coupling, stability, and compatibility with various tool configurations.

“Wedge osteotomy blade” refers to a tool, apparatus, or device configured to engage with an oscillation handpiece for performing wedge-shaped bone resections (“bone wedge”) or wedge osteotomies. A wedge osteotomy blade includes a cutting edge with a width or thickness that increases progressively across the cut face.

A wedge osteotomy blade operates by oscillating through a defined arc, interacting with a bone or other target object to remove material and simultaneously form a wedge-shaped resection. The progressive increase in thickness along the cut face allows the wedge osteotomy blade to create an osteotomy with diverging planes in a single pass, forming a wedge shape that may facilitate correction of alignment, angular deformities, or structural modification of bones.

The wedge osteotomy blade may include a plurality of cutting teeth or similar cutting features that engage with the target object through cutting, abrading, or scraping actions. These cutting features may vary in thickness or geometry to control the final wedge shape produced in the target object, providing the user precision and control over the extent of bone removal and angle of correction.

A wedge osteotomy blade may be adaptable to various powered oscillation handpieces, including those designed for general-purpose oscillating blades or specialized surgical instruments. The blade's geometry may be tailored for specific surgical procedures, from aggressive wedge formation to fine precision reshaping, allowing surgeons or users to achieve desired clinical outcomes efficiently and accurately. (Defined in conjunction with ChatGPT 4.5 Version, Mar. 18 , 2025).

“Powered oscillation handpiece” refers to a motor-driven surgical instrument designed to generate repeated angular displacement about a pivot axis or axis of rotation for use in osteotomies and other precision cutting, shaping, or material removal procedures. The handpiece transmits oscillatory motion to an attached tool—such as a rasp blade, saw blade, burr, or other cutting instrument—allowing for controlled engagement with bone or other surgical targets.

A powered oscillation handpiece may incorporate electromechanical, pneumatic, or other drive mechanisms to provide variable-speed oscillation, enabling precise control over depth, speed, and force of material removal. The device is designed for compatibility with various surgical tool attachments, including those used for bone shaping, contouring, resection, or controlled excision.

In certain embodiments, the powered oscillation handpiece may include ergonomic handling features, vibration-dampening structures, or integrated cooling systems to enhance surgical precision and reduce procedural fatigue. (© ChatGPT 4o Version, Modified, accessed chat. openai. com/chat March 10, 2025).

As used herein, “side” refers to a structure or part of a structure including but not limited to one of a longer bounding surfaces or lines of an object especially contrasted with the ends, a line or surface forming a border or face of an object, either surface of a thin object, a bounding line or structure of a geometric figure or shape, and the like. (search “side” on Merriam-Webster. com. Merriam-Webster, 2021. Web. 3 Aug. 2021. Modified.)

A side can also refer to a geometric edge of a polygon (two-dimensional shape) and/or a face or surface of a polyhedron (three-dimensional shape). (Search “side” on Wikipedia. com Jul. 21, 2021. CC-BY-SA 3.0 Modified. Accessed Aug. 03, 2021.)

Side can also refer to a location on a structure. For example, a side can be a location on a structure at, or near, a furthest position away from a central axis of the structure. As used herein, the term “side” can include one or more modifiers that define and/or orient and/or distinguish the side of an object from others based on based on where and/or how the object is deployed within or in relation to a second object.

For example, in the context of an implant or tool for use with a patient, sides of the implant may be labeled based on where the sides are relative to the patient when the implant is deployed. As one example, an “anterior side” of an implant, instrument, anatomical structure, or other structure refers to a side that is anterior to other sides of the structure in relation to a patient when the structure is deployed in the patient. As another example, in the context of an instrument used with a patient, sides of the instrument may be labeled based on where the sides are when the instrument is being used for its purpose. As one example, a “front side” of an instrument refers to a side that is facing a user of the instrument when the instrument is in use.

As used herein, “feature” refers to a distinctive attribute or aspect of something. (Search “feature” on google.com. Oxford Languages, 2021. Web. 20 Apr. 2021.) A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “rasp feature,” “attachment feature,” “alignment feature,” “securing feature,” “placement feature,” “protruding feature,” “engagement feature,” “disengagement feature,” “resection feature”, “guide feature”, “alignment feature,” and the like.

“Bone cutting feature” refers to a structural element, portion, or part of a device, object, apparatus, or instrument specifically configured, designed, or engineered to engage, cut, abrade, shape, resect, or otherwise modify bone tissue or bone-like materials. A bone cutting feature may include cutting edges, cutting teeth, rasp features, abrasive surfaces, saw blades, wedge structures, or similar elements capable of removing or reshaping bone. The bone cutting feature may vary in geometry, dimensions, or characteristics depending on factors such as intended surgical or medical application, the type or condition of bone tissue targeted, and desired clinical outcomes. Bone cutting features may be found on various components, instruments, or tools, including, without limitation, wedge osteotomy blades, rasp blades, surgical saws, bone burrs, milling cutters, drills, reamers, surgical implants, or other bone-modifying surgical instruments. (Defined in conjunction with ChatGPT 4.5 Version, Mar. 18, 2025).

“Foot” refers to a structural feature of a device, component, apparatus, or system that extends from and/or is connected to a larger device, component, body, apparatus, system, a leg structure, or the like and is configured to engage, support, contact, modify, or apply force to a surface, object, or medium. A foot can facilitate stability, positioning, material interaction, force transmission, or other functional engagements between the device and an external structure. The term “foot” can encompass any structure performing analogous functions to a biological foot, including engagement, stabilization, movement control, and material modification.

In various embodiments, a foot may comprise one or more surfaces, edges, or contact points that interact with a target object or surface. The foot and/or its surfaces, edges, or contact points may be planar, contoured, curved, segmented, flexible, or rigid, and may be positioned at various angles relative to a leg and/or body of a device, component, apparatus, or system. In certain configurations, the foot may be integrally formed with, removably attached to, or movably connected to a leg and/or device to allow for articulation, pivoting, or controlled motion.

A foot may perform one or more functions, including but not limited to: Load distribution and surface engagement, such as stabilizing a cutting or abrading tool against a material surface; Material modification, such as incorporating rasping, cutting, or abrasive features to remove or reshape material; Force application or transmission, such as controlling contact pressure during oscillation or movement; Pivoting or articulated movement, allowing the foot to adjust dynamically during operation.

Examples of a foot include, but are not limited to, a rasp blade foot configured to oscillate against a bone surface, where a planar inferior side includes rasping features for material removal, a surgical saw foot providing a stable contact surface to guide an oscillating or reciprocating blade, a robotic gripper foot designed to stabilize or engage a surface through pressure or frictional contact, a prosthetic foot that mimics the functionality of a biological foot for weight distribution and movement, or the like. (Defined in conjunction with ChatGPT Version 4o, Mar. 11, 2025).

“Planar” refers to a surface, structure, or feature that extends predominantly along a flat geometric plane. A planar surface may be entirely flat or may include minor variations, curvatures, or surface features, provided that it retains an overall substantially flat configuration relative to an adjacent structure, reference axis, or functional requirement.

In various embodiments, a planar surface may be oriented horizontally, vertically, or at an angle relative to another component. A planar surface may include textured, perforated, or contoured features, such as ridges, grooves, rasp features, or recesses, while maintaining an overall flat form. A planar surface may function as a supporting, contacting, guiding, or engaging surface, depending on the application.

Examples of planar features include a planar inferior surface of a rasp blade foot defining a substantially flat engagement surface with rasps features, a planar guide surface of a surgical cutting tool ensuring controlled movement along a fixed plane, a planar electrode contact area in an electronic device facilitating uniform electrical interaction, and a planar bearing surface in a mechanical assembly allowing for stable load distribution.

As used herein, the term “planar” encompasses surfaces that are predominantly flat in structure while permitting minor deviations, texturing, or functional modifications consistent with their intended use. (© ChatGPT 4o Version, Modified, accessed chat. openai. com/chat Mar. 11, 2025).

“Relief” refers to a feature in a mechanical component that removes material to reduce stress concentrations, minimize friction, facilitate assembly, or improve functionality. A relief may take the form of a recess, groove, cutout, chamfer, or clearance area that prevents unwanted contact, interference, or excessive material buildup. The placement and geometry of a relief may be optimized to enhance mechanical performance, extend component lifespan, and ensure efficient operation within an assembly. In cutting tools and machined components, a relief may be incorporated to reduce cutting forces, prevent excessive wear, and improve chip evacuation. For example, in a cutting tooth, cutting edge, or set of cutting teeth, a relief may refer to a clearance angle or back-off behind the cutting edge, reducing material contact and friction. A relief may also be included in bearing surfaces, shafts, fasteners, or other structural components to allow controlled movement, prevent binding, and accommodate tolerances. (Defined in conjunction with ChatGPT Version 4o, Mar. 13, 2025).

“Evacuation” refers to the process of removing bone debris, bone chips, or other particulate material from the cutting face or set of rasp features of a surgical tool during operation. Evacuation may occur through passive or active mechanisms to enhance cutting efficiency, maintain clear visibility, and reduce heat buildup or clogging. In certain embodiments, evacuation may be facilitated by tooth or rasp feature geometry, including gullet spacing, serrations, or fluting, which directs bone debris away from the cutting face; oscillatory or reciprocating motion, which aids in self-clearing of bone material from engagement surfaces; integrated fluid irrigation systems, such as saline or cooling agents, which assist in flushing debris from the cutting interface; vacuum-assisted suction mechanisms, which actively remove bone particles from the cutting region; openings, ports, or channels specifically designed and/or configured to facilitate evacuation, which may include apertures within the blade, fenestrations in the rasp surface, or grooves along the cutting edge to promote continuous removal of debris. As used herein, “evacuation” encompasses any mechanism or structural design feature that promotes the removal, displacement, or clearing of bone debris from the cutting face or rasp features, whether by mechanical action, fluid assistance, suction, or integrated openings configured for debris management. (Defined in conjunction with ChatGPT Version 4o, Mar. 12, 2025).

“Bone debris” refers to particulate material generated during the cutting, shaping, or modification of bone using surgical instruments such as osteotomy blades, rasp blade, contouring blades, osteotomy rasps, saws, drills, or the like. Bone debris may include bone chips, bone dust, fragments, or other dislodged material resulting from mechanical interaction between a cutting tool or rasping tool or contouring tool and bone tissue. In certain embodiments, bone debris may vary in size, composition, and morphology, depending on factors such as the cutting speed, tool geometry, bone density, and applied force. Bone debris may consist of cortical bone particles, cancellous bone fragments, or a combination thereof, and may accumulate at or near the cutting interface/face, and may affect cutting efficiency, visibility, and/or heat dissipation. As used herein, “bone debris” encompasses any solid byproduct of bone modification that may require evacuation, removal, or management to maintain optimal surgical performance, ensure procedural accuracy, or facilitate bone healing. (Defined in conjunction with ChatGPT Version 4o, Mar. 12, 2025).

“Tooth” or “teeth” refers to a structural feature of a cutting, rasping, or abrading tool that is configured to engage, cut, tear, abrade, or otherwise modify a target material through mechanical interaction. A tooth may be arranged singularly or in a plurality along a cutting edge, along a cutting face, on an abrading surface, and/or within a rasp feature set. A tooth and may be structured, sized, or configured, to optimize material removal efficiency, cutting precision, abrading precision, and/or surface contouring. A tooth may be shaped, arranged, or functionally similar to biological teeth found in animals or humans, but may also encompass artificial or engineered structures specifically designed for cutting, abrading, or shaping applications.

A tooth may include a base, a point, a tip, and/or an edge, a face, at least one side, and/or a thickness measured from one side to an opposite side of the tooth. The base refers to the portion of the tooth that connects to, or is supported by, a blade, a body, a surface, or a substrate. The point refers to the terminal end of the tooth, which may come to a sharp tip, may resemble a chisel, or may taper into an edge to facilitate material engagement. The face of the tooth includes a surface that contacts the target material. A tooth face may have a rake angle, which can influence the cutting, shearing, or abrading interaction of the tooth with the target material. The at least one side or plurality of sides of the tooth define its lateral boundaries and may contribute to the overall cutting or abrading geometry. The thickness of the tooth is measured between opposing sides and may determine the durability, rigidity, and cutting efficiency of the tooth. A tooth height is measured from the point to the base.

A tooth may be made from a variety of materials, including but not limited to stainless steel, titanium, carbide, ceramic, composite materials, polymers, biocompatible materials, or other engineered substances selected for durability, cutting performance, and wear resistance. A tooth may vary in design based on application-specific requirements and may include serrated, chisel-shaped, hooked, beveled, offset, or other specialized geometries to optimize cutting, abrading, or contouring operations. (Defined in conjunction with ChatGPT Version 4o, Mar. 12, 2025).

“Wedge osteotomy” refers to an osteotomy procedure in which one or more wedges are used as part of the procedure. Generally, wedge osteotomies can be of one of two types, open wedge and closing wedge. The type of osteotomy refers to how the procedure changes the relation between two parts of a bone involved in the osteotomy. In an open wedge osteotomy a wedge of bone or graft or other material is inserted in between two parts of a bone. Consequently, a wedge shape is “opened” in the bone. In a close/closed wedge osteotomy or closing wedge osteotomy, a wedge of bone is removed from a bone. Consequently, a wedge shape formed in the bone is “closed.”

“Bone Wedge” refers to a geometric shape of one or more bones characterized by having two flat, planar, and/or inclined sides or surfaces that converge to form an edge. A bone wedge resembles a triangular prism, with one end wider or thicker than the other. (© ChatGPT August 3 Version, Modified, accessed chat. openai. com/chat Sept. 28, 2023). In certain implementations, the edge formed by the two converging sides is within a bone or set of bones from which the bone wedge is formed. In other implementations, the edge formed by the two converging sides is outside of a bone or set of bones from which the bone wedge is formed.

“Wedge Angle” refers to an angle measured between two surfaces or planes of a wedge shape. A wedge angle can also be an angle between two sides of a wedge shape.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. (Search “coupling” on Wikipedia. com Jul. 26, 2021. CC-BY-SA 3.0 Modified. Accessed Jul. 27, 2021.) A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

As used herein, “implant” refers to a medical device manufactured to replace a missing biological structure, support a damaged biological structure, or enhance an existing biological structure. Often medical implants are man-made devices, but implants can also be natural occurring structures. The surface of implants that contact the body may be made of, or include a biomedical material such as titanium, cobalt chrome, stainless steel, carbon fiber, another metallic alloy, silicone, polymer, Synthetic polyvinyl alcohol (PVA) hydrogels, biomaterials, biocompatible polymers such as PolyEther Ether Ketone (PEEK) or a polylactide polymer (e.g. PLLA) and/or others, or apatite, or any combination of these depending on what is functional and/or economical. Implants can have a variety of configurations and can be wholly, partially, and/or include a number of components that are flexible, semiflexible, pliable, elastic, supple, semi-rigid, or rigid. In some cases, implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants can be used to treat bone fractures, osteoarthritis, scoliosis, spinal stenosis, discomfort, and pain. Examples of orthopedic implants include, but are not limited to, a wide variety of pins, rods, screws, anchors, spacers, sutures, all-suture implants, ball all-suture implants, self-locking suture implants, cross-threaded suture implants, plates used to anchor fractured bones while the bones heal or fuse together, and the like. (Search “implant (medicine)” on Wikipedia. com May 26, 2021. CC-BY-SA 3.0 Modified. Accessed Jun. 30, 2021.)

“Fixation,” “fixation device,” “fastener,” or “fastener system” refers to an apparatus, instrument, structure, device, component, member, system, assembly, step, process, or module that is structured, configured, designed, arranged, or engineered to connect, join, engage, or couple two or more structures, either permanently or temporarily. The connected structures may be manmade and/or biological and may include hard tissues such as bone, teeth, or similar materials, as well as soft tissues such as ligaments, cartilage, tendons, or similar biological structures. In certain embodiments, fixation or fastening serves to secure two structures in a desired position and/or orientation, redistribute load or stress, maintain a desired level of tension or compression, and/or reduce relative motion between connected components.

A fixation device or fastener may be made of metal, plastic, composite materials, metal alloys, plastic composites, biocompatible materials, biodegradable materials, or other suitable materials. In some embodiments, a fixation device or fastener may be part of a fastener system that includes two or more structures that work together to function as a fastening mechanism. For example, a fastener system may include a rod or shaft having external threads and an opening or bore within another structure having corresponding internal threads configured to engage the external threads of the rod or shaft.

Fixation devices and fasteners may be used in internal or external fixation applications and may include, but are not limited to: screws, bone screws, set screws, rivets, bolts, nails, pins, Kirschner wires (K-wires), anchors, bone anchors, plates, bone plates, posts, thumb screws, nuts, intramedullary nails, rods, implants, sutures, soft sutures, soft anchors, tethers, interbody cages, fusion cages, staples, bone staples, hook-and-loop mechanisms, snaps, and similar structures. In certain embodiments, a fastener may include an adjective identifying an object or structure that the fastener is particularly configured, designed, or engineered to engage, connect, or couple with. For example, a “bone fastener” may refer to an apparatus for joining or connecting one or more bones, one or more bone portions, soft tissue and a bone or bone portion, hard tissue and a bone or bone portion, or an apparatus and a bone or portion of bone.

In certain embodiments, a fastener may be a temporary fastener, meaning that the fastener is configured to serve a fastening function for a relatively short period of time. A temporary fastener may be used until another procedure or operation is completed and/or until a particular event occurs. A temporary fastener may be designed for removal by a user or may be configured to disengage due to an external event, structural change, or mechanical interaction. (Defined in conjunction with ChatGPT Version 4o, Mar. 12, 2025).

“Tissue” refers to a structure that makes up a one or more anatomical structures of a patient. Tissue can be soft tissue or hard tissue. “Soft tissue” refers to tissue of a patient. Examples of soft tissue include but are not limited to skin, ligament, tendon, fascia, fat muscle, fibrous tissue, blood vessels, lymph vessels, brain tissue, and/or nerves. “Hard tissue” refers to any human tissue that is not soft tissue. Examples of hard tissue include bone, teeth, tooth enamel, dentin, cementum, cartilage, or the like.

As used herein, “osteotomy procedure” or “surgical osteotomy” or “osteotomy” refers to a surgical operation in which one or more bones are cut to shorten or lengthen them or to change their alignment. The procedure can include removing one or more portions of bone and/or adding one or more portions of bone or bone substitutes. (Search “osteotomy” on Wikipedia.com Feb. 3, 22, 2021. CC-BY-SA 3.0 Modified. Accessed Feb. 15, 2022.) As used herein, “patient-specific osteotomy procedure” refers to an osteotomy procedure that has been adjusted, tailored, modified, or configured to specifically address the anatomy, physiology, condition, abnormalities, needs, or desires of a particular patient. In certain aspects, one patient-specific osteotomy procedure may be useable in connection with only one patient. In other aspects, one patient-specific osteotomy procedure may be useable with a number of patients having a particular class of characteristics. In certain aspects, a patient-specific osteotomy procedure may refer to a non-patient-specific osteotomy procedure that includes one or more patient-specific implants and/or instrumentation. In another aspects, a patient-specific osteotomy procedure may refer to a patient-specific osteotomy procedure that includes one or more patient-specific implants, patient-specific surgical steps, and/or patient-specific instrumentation.

As used herein, a “base” refers to a main or central structure, component, or part of a structure. A base is often a structure, component, or part upon which, or from which other structures extend into, out of, away from, are coupled to, or connect to. A base may have a variety of geometric shapes and configurations. A base may be rigid or pliable. A base may be solid or hollow. A base can have any number of sides. In one embodiment, a base may include a housing, frame, or framework for a larger system, component, structure, or device. In certain embodiments, a base can be a part at the bottom or underneath a structure designed to extend vertically when the structure is in a desired configuration or position. Certain bones such as a metatarsal bone can include a base as one structural component of the bone.

“Visibility” refers to a state of feature, aspect, structure, or thing being capable of being observed, seen, or perceived.

As used herein, a “protrusion” refers to a structure or portion of a structure that protrudes or extends from at least one other structure such as a surface of the at least one other structure. Generally, the other structure is connected to, or in contact with, the protrusion.

“Set” refers to a collection of objects. A set can have zero or more objects in the collection. Generally, a set includes one or more objects in the collection.

“Fusion” refers to a natural process of bone growth and generation in which two separate bones and/or bone fragments grow together as new bone grows when the two separate bones and/or bone fragments contact each other. Often, fusion is facilitated by compression of the two separate bones and/or bone fragments towards each other.

As used herein, a “resection” refers to a method, procedure, or step that removes tissue from another anatomical structure or body. A resection can include an osteotomy that cuts through a bone or other tissue because the osteotomy still removes at least a minimal amount of tissue. A resection is typically performed by a surgeon on a part of a body of a patient. A resection is one type of osteotomy. (Search “surgery” on Wikipedia.com May 26, 2021. CC-BY-SA 3.0 Modified. Accessed May 26, 2021.) Resection may be used as a noun or a verb. In the verb form, the term is “resect” and refers to an act of performing, or doing, a resection. Past tense of the verb resect is “resected”.

As used herein, a “condition” refers to a state of something with regard to its appearance, quality, or working order. In certain aspects, a condition may refer to a patient's state of health or physical fitness or the state of health or physical fitness of an organ or anatomical part of a patient. In certain embodiments, a condition may refer to an illness, pain, discomfort, defect, disease, or deformity of a patient or of an organ or anatomical part of a patient. (Search “condition” on wordhippo.com. WordHippo, 2021. Web. Accessed 8 Dec. 2021. Modified.)

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region. “Malrotation” refers to a condition in which a part, typically a part of a patient's body has rotated from a normal position to an unnormal or uncommon position.

“Cortical bone” refers to a type of bone tissue. Cortical bone is a type of bone tissue typically found between an external surface of a bone and an interior area of the bone. Cortical bone is more dense and typically stronger structurally than other types of bone tissue. “Cortical surface” refers to a surface of cortical bone.

“Cortex” refers to an area of bone that extends from an external surface of the bone towards a center part of the bone. The cortex is typically comprised of cortical bone.

“Cutting tool” refers to any tool that can be used to cut or resect another object. In particular, a cutting tool can refer to a manual or power tool for cutting or resecting tissue of a patient. Examples of cutting tools include, but are not limited to, a burr, an oscillating saw, a reciprocating saw, a grater saw, a drill, a mill, a side-cutting burr, or the like.

As used herein, “cutting edge” refers to an edge designed, adapted, configured, or engineered to facilitate cutting into another structure or object to remove parts of the structure or object and/or to cut through the structure or object. Examples of a cutting edge, include, but are not limited to, an edge of a knife, tooth, blade, saw, or the like.

“Contour” refers to an outline representing or bounding a shape or form of an object. Contour can also refer to an outside limit of an object, area, or surface of the object. (Search “contour” on wordhippo.com. WordHippo, 2023. Web. Modified. Accessed 13 Jun. 2023.)

“Surgical procedure” refers to a process, method, or series of steps performed using medical instruments, devices, or techniques to diagnose, treat, address, modify, repair, or remove biological tissues in a controlled manner. A surgical procedure may be performed on soft tissues, hard tissues, or a combination thereof and may involve cutting, reshaping, rasping, contouring, repositioning, removing, repairing, grafting, or otherwise altering tissues to achieve a desired medical or functional outcome. A surgical procedure may be conducted through open surgery, minimally invasive techniques, robotic-assisted methods, or other medical approaches depending on the specific application.

In the context of osteotomy surgical procedures of the foot, a surgical procedure may include, but is not limited to, distal metatarsal osteotomy, proximal metatarsal osteotomy, diaphyseal osteotomy, Chevron osteotomy, Scarf osteotomy, Ludloff osteotomy, Akin osteotomy, Weil osteotomy, Cotton osteotomy, Evans osteotomy, Dwyer osteotomy, Lapidus procedure, closing wedge osteotomy, opening wedge osteotomy, and the like. (Defined in conjunction with ChatGPT Version 4o, Mar. 12, 2025).

As used herein, “edge” refers to a structure, boundary, or line where an object, surface, or area begins or ends. An edge can also refer to a boundary or perimeter between two structures, objects, or surfaces. An edge can also refer to a narrow part adjacent to a border. (search “edge” on Merriam-Webster.com. Merriam-Webster, 2021. Web. 3 Aug. 2021. Modified.) In certain embodiments, an edge can be a one dimensional or a two-dimensional structure that joins two adjacent structures or surfaces. Furthermore, an edge may be at a perimeter of an object or within a perimeter or boundary of an object.

“Bone fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

“Cut surface” or “cut face” refers to a surface of an object that is created or formed by the removal of one or more parts of the object that includes the original surface. Cut surfaces or cut faces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface or cut face can include manual, mechanical, motorized, hydraulic, automated, robotic, and the like.

“Nonuniform thickness” refers to a characteristic of an object, material, or structural component having a thickness that varies or changes along its length, width, or surface area. Nonuniform thickness may include gradual or abrupt variations, changes in dimensions, or thickness profiles designed to meet specific structural, functional, or operational requirements. The variation in thickness may be defined, characterized, or measured relative to one or more reference dimensions, axes, surfaces, or other geometric or functional features of the object or structure. Objects exhibiting nonuniform thickness can include, without limitation, cutting blades, wedges, mechanical parts, surgical implants, wedge osteotomy blades, or other components engineered for specific performance or application needs. (Defined in conjunction with ChatGPT 4.5 Version, Mar. 18, 2025).

“Width” refers to a dimension, measurement, or characteristic of an object, material, or structural component that extends across or perpendicular to a defined length, axis, or reference feature. Width may be uniform or varied (nonuniform) and can be defined, characterized, or measured relative to one or more reference dimensions, axes, surfaces, or other geometric or functional features of the object or structure. Objects or components having width include, without limitation, cutting blades, wedges, mechanical parts, surgical implants, implants, wedge osteotomy blades, or other engineered elements designed for specific performance or application needs. (Defined in conjunction with ChatGPT 4.5 Version, Mar. 18, 2025).

“Height” refers to a dimension or characteristic of an object, material, or structural component that extends vertically, perpendicularly, or in an orientation distinct from length or width, relative to a defined axis, plane, or reference feature. Height may be uniform or vary (nonuniform) and can be defined, characterized, or measured relative to one or more reference dimensions, axes, surfaces, or other geometric or functional features of the object or structure. Objects or components having height include, without limitation, cutting blades, wedges, mechanical parts, surgical implants, tools, wedge osteotomy blades, or other components engineered for specific performance or application needs. (Defined in conjunction with ChatGPT 4.5 Version, Mar. 18, 2025).

“Tip” refers to a feature, portion, or part of a tooth or similar cutting element of a tool, apparatus, or structural component configured to engage, cut, abrade, or otherwise interact with a target material or surface. The tip typically represents the leading, terminal, or distal portion of the tooth and may include a sharp point, edge, surface, or apex specifically configured or designed to facilitate penetration, cutting, or material removal. The geometry and characteristics of a tip may vary based on the intended application, material to be cut, and desired cutting performance. A tip may be defined, characterized, or measured relative to one or more reference dimensions, axes, planes, or geometric or functional features of the tooth or associated cutting structure. Components having a tip include, without limitation, cutting teeth, abrading teeth, rasping features, blades, drills, cutters, cutting features, wedge structures, or other engineered cutting elements utilized in mechanical, surgical, or manufacturing processes. (Defined in conjunction with ChatGPT 4.5 Version, Mar. 18, 2025).

“Face” refers to a surface, portion, or region of a cutting tooth or similar cutting structure designed or configured to interact with, engage, cut, abrade, or remove material from a target object. The face typically extends from a cutting edge or tip of the tooth toward a base or body of the tooth, and may be angled, contoured, planar, or otherwise shaped to achieve specific cutting or abrading characteristics. The geometry and characteristics of a face may vary based upon factors such as desired cutting efficiency, material to be cut, and overall tooth performance. A face may be defined, characterized, or measured relative to one or more reference dimensions, axes, surfaces, or geometric or functional features of the tooth or structure on which it resides. Components incorporating teeth having faces include, without limitation, wedge osteotomy blades, rasp blades, saw blades, milling cutters, surgical cutting tools, machining tools, and other cutting or abrading instruments configured for material shaping, modification, or removal. (Defined in conjunction with ChatGPT 4.5 Version, Mar. 18, 2025).

“Cut face” refers to a surface, region, or portion of a cutting tool that is defined by and/or results from the arrangement, orientation, and interaction of one or more cutting teeth, cutting edges, and/or cutting features. The cut face may be formed by a collective engagement of cutting elements (or subset of cutting elements) with a target material, influencing the characteristics of material removal, surface finish, and cutting performance. The geometry, shape, and configuration of the cut face may vary based on factors such as the positioning and spacing of cutting teeth, the rake and engagement angles of the cutting features, how a cutting tool is operated and/or the intended cutting application. Tools or instruments incorporating a cut face include, without limitation, wedge osteotomy blades, rasp blades, saw blades, milling cutters, surgical cutting tools, burrs, and other cutting or abrading instruments configured for controlled material removal. (Defined in conjunction with ChatGPT 4.5 Version, Mar. 18, 2025).

“Rake angle” refers to the angle formed between the face of a cutting tooth and a reference line or plane that is perpendicular to the direction of cutting or relative to an axis or plane associated with the tooth or the cutting tool. The rake angle can positively influence the cutting performance, efficiency, and effectiveness of a cutting tooth by affecting factors such as cutting force, chip formation, surface finish, and tool longevity. The rake angle may be positive, negative, or neutral, depending on the intended application, the type of material being cut, and the desired cutting behavior. Teeth having a rake angle can be incorporated into various components, including, without limitation, wedge osteotomy blades, saw blades, rasp blades, milling cutters, drill bits, surgical instruments, and other cutting, shaping, or abrading tools. The rake angle can be defined, characterized, or measured relative to one or more reference planes, axes, surfaces, or dimensions associated with the tooth or the overall cutting tool. (Defined in conjunction with ChatGPT 4.5 Version, Mar. 18, 2025)

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening.” As one example, a “fastener opening” refers to an “opening” adapted, configured, designed, or engineered to accept or accommodate a “fastener.”

“Channel” refers to a structural feature, groove, recess, pathway, opening, or passage formed in and/or between one or more teeth, cutting features and/or other structures of a cutting tool. A channel may be configured, designed, or engineered to facilitate functions including, but not limited to, evacuation or removal of debris, guiding or redirecting chips or debris away from the cutting surface, improving cutting efficiency, reducing frictional forces, enhancing cooling or lubrication, or otherwise optimizing the cutting or abrading performance of the tool. Channels may vary in shape, size, depth, and configuration based on the intended application, the nature of the material being cut, and desired tool performance characteristics. Components or tools featuring teeth that include channels may include, without limitation, wedge osteotomy blades, rasp blades, saw blades, milling cutters, burrs, drills, surgical cutting tools, and similar cutting or abrading instruments. (Defined in conjunction with ChatGPT 45 Version, Mar. 18, 2025).

“Engagement angle” refers to an angle formed between a tooth, cutting element, or bone cutting feature of a cutting tool and a target material or surface at the point or region of initial or sustained interaction, engagement, or contact. The engagement angle can influence cutting efficiency, performance characteristics, depth of cut, force requirements, precision, and quality of the material removal process. The engagement angle may be defined, characterized, or measured relative to one or more reference axes, planes, tooth surfaces, tool orientations, or other suitable geometric or functional reference features. Teeth or cutting elements with defined engagement angles may be present in tools or instruments including, without limitation, wedge osteotomy blades, rasp blades, saw blades, milling cutters, drills, burrs, surgical cutting instruments, or other similar tools used for mechanical, surgical, manufacturing, or material-shaping applications. (Defined in conjunction with ChatGPT 4.5 Version, Mar. 18, 2025).

“Cut line” refers to a defined path, boundary, trajectory, or line formed or intended to be formed by a cutting tool as it engages, penetrates, separates, or removes material from a target object or surface. The cut line may represent the region or boundary of separation created by one or more teeth, edges, or cutting features of the tool. The characteristics, quality, precision, or dimensions of the cut line can depend upon factors such as the geometry of the cutting tool, the configuration and orientation of the teeth or cutting elements, the motion or operation of the tool, and the properties of the target material. Cutting tools or instruments configured to form or define a cut line include, without limitation, wedge osteotomy blades, rasp blades, saw blades, milling cutters, surgical cutting instruments, blades, drills, burrs, or other similar cutting, abrading, or material-shaping tools. (Defined in conjunction with ChatGPT 4.5 Version, Mar. 18, 2025).

“Vertex” refers to a point at which lines, structures, trajectories, or pathways intersect. (Search “vertex” on wordhippo.com. WordHippo, 2023. Web. Modified. Accessed 13 Jun. 2023.)

“Apex” refers to a point, region, or junction within a wedge osteotomy where two intersecting planes of the osteotomy converge, forming the narrowest portion of the resected wedge. The apex may define the focal point of angular correction, structural modification, or bone realignment resulting from the osteotomy. The position, orientation, and geometry of the apex may vary based on surgical technique, bone morphology, and the intended clinical outcome. The apex may be characterized or measured relative to one or more reference axes, planes, or anatomical landmarks to ensure precision in bone resection and correction. Instruments or tools configured to create an apex in a wedge osteotomy include, without limitation, wedge osteotomy blades, saw blades, bone-cutting guides, surgical cutting instruments, or other devices used in orthopedic or reconstructive procedures. (Defined in conjunction with ChatGPT 4o Version, Mar. 18, 2025).

“Single pass” refers to a mode of operation in which an oscillating wedge osteotomy blade completes a full bone resection, cut, or wedge osteotomy in one continuous motion or stroke without requiring multiple sequential passes or repeated repositioning of the blade. A single pass may be facilitated by the geometry of the blade, including a progressively increasing thickness across the cut face, cutting teeth orientation, or oscillatory motion, allowing for controlled separation of bone segments in a single engagement. Effectiveness of a single pass may depend on factors such as blade design, oscillation amplitude, bone density, and applied force. Instruments or tools capable of performing a single pass osteotomy include, without limitation, oscillating wedge osteotomy blades, surgical saws, bone-cutting instruments, and similar surgical devices configured for precision bone modification. (Defined in conjunction with ChatGPT 4o Version, Mar. 18, 2025).

The present disclosure discloses an apparatus, system, and method for forming precise and controlled wedge osteotomies in one or more bones. The present disclosure provides a wedge osteotomy blade configured to connect to a powered oscillation handpiece. In certain embodiments, the blade includes a plurality of cutting teeth having a nonuniform thickness across a width of the cutting edge that allow a surgeon to create single-pass bone wedge resections with improved efficiency and accuracy. In another embodiment, the blade includes a plurality of cutting teeth wherein a thickness (or length) of the cutting features of the teeth varies across the cutting edge allowing a surgeon to create single-pass bone wedge resections with improved efficiency and accuracy. In another embodiment, the blade includes a plurality of cutting teeth wherein teeth thickness increases laterally across a cutting edge of the blade allowing a surgeon to create single-pass bone wedge resections with improved efficiency and accuracy. In another embodiment, the blade includes a plurality of cutting teeth wherein the plurality of cutting teeth have a thickness that increases from a first lateral side to a second lateral side across a width of the cutting edge of the blade allowing a surgeon to create single-pass bone wedge resections with improved efficiency and accuracy. These configurations enable consistent cut geometry while reducing the risk of misalignment or over-resection associated with multi-pass techniques.

Advantageously, the present disclosure includes one or more wedge osteotomy blade designs with a cutting edge and/or cutting features that facilitates a single-pass osteotomy, including wedge resections, particularly in small bones where precision is critical. A widening, flared, and/or wedge-shaped (and/or elongated) cutting feature(s) facilitate forming a wedge osteotomy allowing a surgeon to make highly accurate wedge angles for wedge osteotomy cuts. The ability to create a wedge osteotomy in a single pass helps maintain bone stock, reduces surgical time, and improves the reliability of angular correction. Alternatively, or in addition, a surgeon may start with a wedge osteotomy blade forming a smaller wedge angle and This allows the surgeon to execute complex bone resection procedures with confidence and control, while minimizing complications related to bone loss or angular deviation.

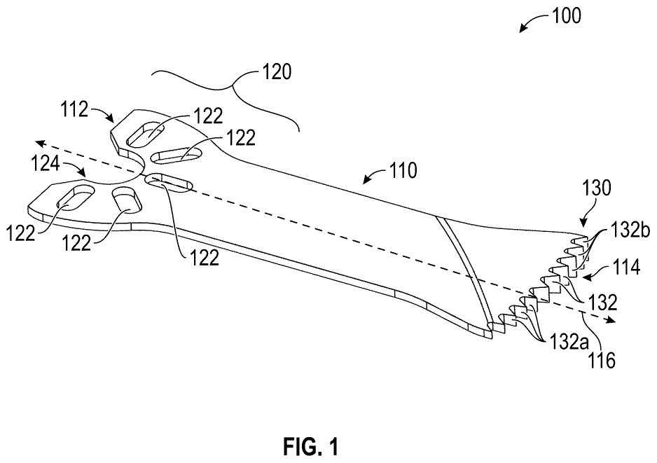

FIG. 1 is a perspective view of an apparatus 100 for forming a wedge osteotomy according to one embodiment. The apparatus 100 may include, among other features and/or structures, a body 110, an oscillation mount 120, and a cutting edge 130.

The body 110 provides a primary structural component of the apparatus 100 and may define a generally planar, contoured, or shaped structure configured to support and connect other features of the apparatus 100. The body 110 may serve as a mounting region for the oscillation mount 120. In another embodiment, the body 110 may connect or may be connectable to the oscillation mount 120.

The geometry, thickness, and overall configuration of the body 110 may vary depending on factors such as the intended application, the type of oscillation handpiece used, and the desired interaction with the bone surface. In some embodiments, the body 110 may incorporate reinforcement structures, openings, surface treatments, and/or other design elements to enhance performance, stability, or material removal efficiency. The body 110 may be formed from various biocompatible or wear-resistant materials, including but not limited to metals, ceramics, polymers, or composite materials. In certain embodiments, the body 110 includes a proximal end 112, a distal end 114, and a longitudinal axis 116 that extends between the proximal end 112 and the distal end 114.

FIG. 1 illustrates one example configuration for an oscillation mount 120. The oscillation mount 120 may be substantially flat and may include a plurality of receiving slots 122 and a center clearance opening 124. The oscillation mount 120 may be clamped by a mount of a powered oscillation handpiece. A spring, not shown, may be disposed within the mount to provide a clamping force against the oscillation mount 120. One or more of the slots 122 may receive an engaging tine (not shown) of the powered oscillation handpiece. A wide variety of mount configurations are found in commercial use. While the inclusion of an oscillation mount 120 is necessary for the use of the presently disclosed apparatus 100, the particular mount configuration of the oscillation mount 120 selected may not be critical to the disclosed apparatus 100, so long as the mount configuration of the oscillation mount 120 is compatible with a mount of the powered oscillation handpiece with which the apparatus 100 is used.

In the illustrated embodiment, the oscillation mount 120 is at the proximal end 112. The oscillation mount 120 is configured to securely couple the apparatus 100 to a powered oscillation handpiece. Advantageously, the oscillation mount 120 is configured to enable use of the apparatus 100 in common powered oscillation handpieces used in the industry. Thus, a special tool is not needed to use the apparatus 100.

The cutting edge 130 engages with a target material, such as bone, cartilage, soft tissue, or the like, to facilitate resection, cutting, abrading, scoring, shaping, removal of material, or the like. The cutting edge 130 is positioned at or near the distal end 114 of the body 110.

The cutting edge 130 may perform these functions through mechanical interactions including slicing, tearing, scraping, shearing, grinding, or the like. In various embodiments, the cutting edge 130 may include one or more cutting features, such as a plurality of cutting teeth 132, serrations, ridges, projections, abrasive structures, or the like. The geometry, spacing, orientation, material, or the like of the cutting edge 130 and its associated features may be selected or configured to optimize performance for a particular procedure, anatomical site, clinical objective, or the like.

In some embodiments, the cutting edge 130 is integrally formed with the body 110, while in other embodiments, the cutting edge 130 may be separately formed and affixed by bonding, welding, molding, mechanical attachment, or the like. The cutting edge 130 may also vary in sharpness, surface texture, profile, or the like along its length and/or width and/or thickness to enable differential engagement with target materials and to support various surgical techniques, use cases, desired outcomes, or the like.

The cutting teeth 132 may be formed from a variety of materials selected to optimize cutting efficiency, durability, biocompatibility, or the like. In some embodiments, the cutting teeth 132 are formed from metallic materials such as stainless steel, titanium, cobalt-chromium alloys, or the like. In other embodiments, the cutting teeth 132 may include ceramic materials, polymeric materials, composite materials, or the like. The cutting teeth 132 may be integrally formed from the same material as the body 110, or they may be separately formed and joined using bonding, welding, press-fitting, overmolding, or the like. In certain embodiments, the cutting teeth 132 may be coated or treated to enhance performance, such as with a diamond-like carbon (DLC) coating, titanium nitride (TiN) coating, abrasive grit layer, chemical etching, or the like. The selected material composition may vary depending on the intended application, desired wear resistance, compatibility with powered instruments, sterilization requirements, or the like.

In some embodiments, the cutting teeth 132 may have a nonuniformity, such as a nonuniform thickness across the width of the cutting edge 130. For example, one or more cutting teeth 132 positioned on a first lateral side of the cutting edge 130 may have a different thickness (i.e., width) than one or more cutting teeth 132 on an opposite lateral side. This nonuniformity may be used to influence the wedge geometry created during an osteotomy, control the directional engagement of the blade with a target material, or provide feedback to a surgeon based on resistance felt during cutting. In certain configurations, thicker cutting teeth may be positioned laterally opposite thinner teeth to provide asymmetric cutting dynamics, improved clearance, or enhanced cutting trajectory control.

In other embodiments, the cutting teeth 132 and/or cutting features may vary in height, width, length, mass, or material properties across the width of the cutting edge 130. For example, the teeth on one lateral side may be longer or extend further superiorly or inferiorly than those on the opposite side, thereby contributing to a sloped or wedge-shaped resection in a single pass. For example, in the illustrated embodiment, teeth 132a are thinner, or not as thick, as teeth 132b, which are wider or more thick than teeth 132a. This difference in dimension can serve to form a wedge osteotomy as the apparatus 100 oscillates in the powered oscillation handpiece during operation.

Alternatively, or in addition, in some configurations, one side of the cutting edge 130 may include more densely spaced teeth, while the other side has fewer or more widely spaced teeth. Asymmetry may also be achieved by varying tooth profile, edge sharpness, rake angle, or cutting angle. These variations may be selected based on the desired resection geometry, cutting efficiency, anatomical orientation, or other clinical considerations. Combinations of these features may be incorporated in a single device to create predictable and efficient directional resection profiles.

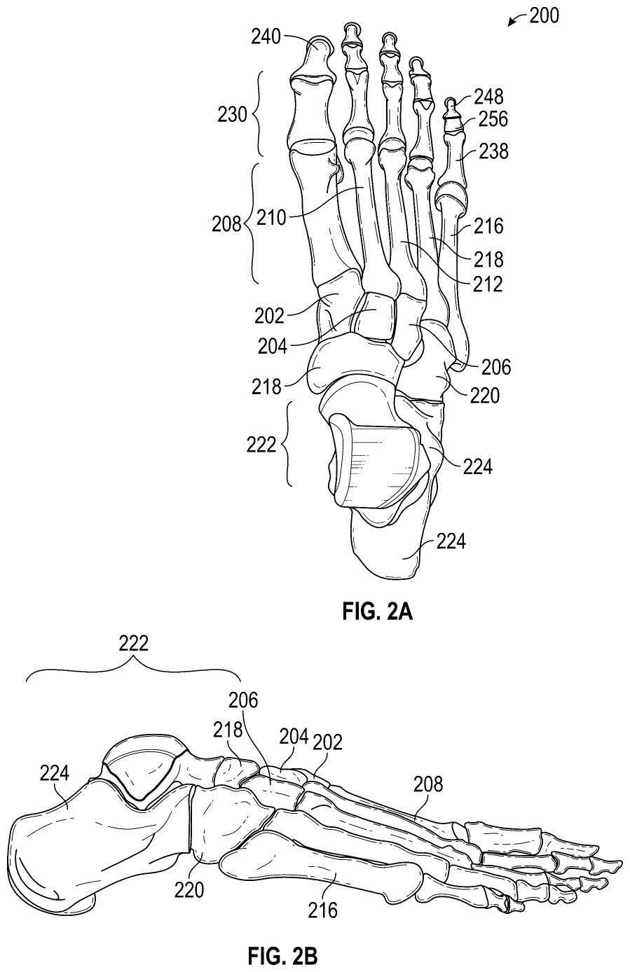

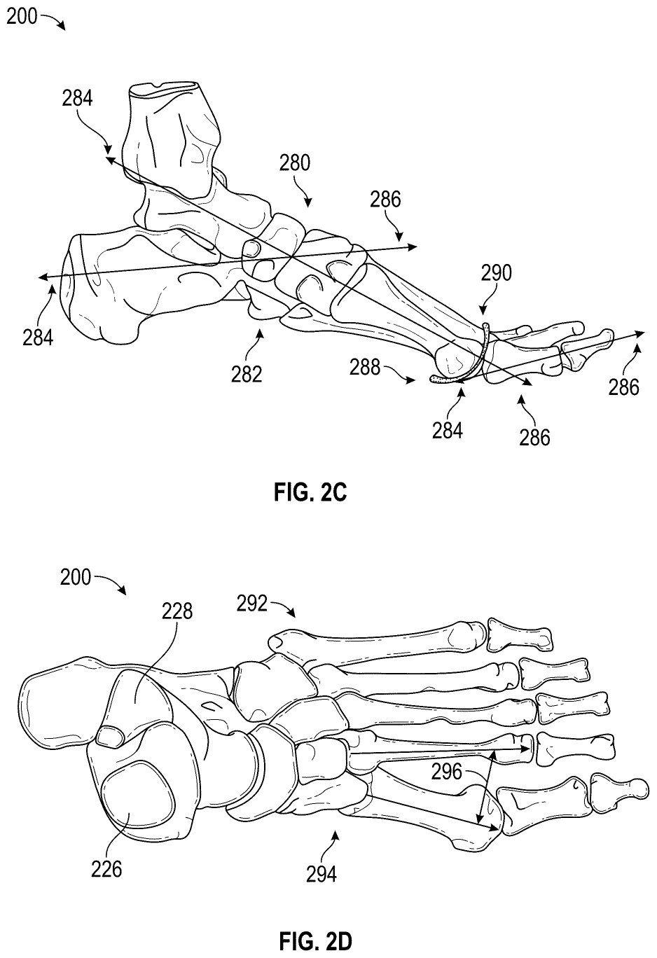

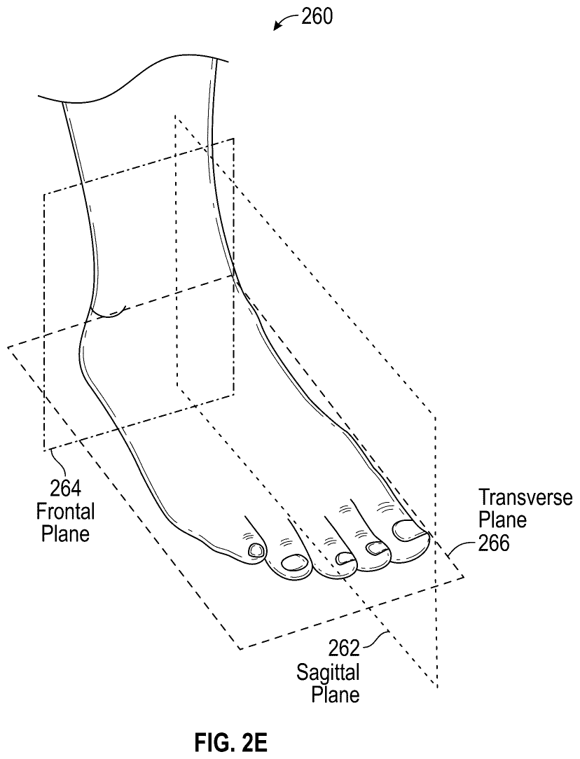

FIG. 2A is a perspective dorsal view of a foot 200. The foot 200 may have a medial cuneiform 202, an intermediate cuneiform 204, lateral cuneiform 206, a first metatarsal 208, a second metatarsal 210, third metatarsal 212, fourth metatarsal 214, fifth metatarsal 216, navicular 218, cuboid 220, talus 222, and calcaneus 224, among others. The medial cuneiform 202 and the intermediate cuneiform 204 may be joined together at a first metatarsocuneiform joint, and the first metatarsal 208 and the second metatarsal 210 may be joined together at a second metatarsocuneiform joint. The foot 200 includes a set of proximal phalanges numbered first through fifth (230, 232, 234, 236, 238) and a set of distal phalanges numbered first through fifth (240, 242, 244, 246, 248) and a set of middle phalanges numbered second through fifth (250, 252, 254, 256).

FIG. 2B is a perspective lateral view of a foot 200, with bones of the foot labeled.

FIG. 2C is a perspective medial view of a foot illustrating a dorsal side 280 and a plantar side 282. The foot 200, as illustrated, may have a tibia 226 and a fibula 228, among others. Dorsal refers to the top of the foot. Plantar refers to the bottom of the foot. Proximal 284 is defined as “closer to the primary attachment point”. Distal 286 is defined as “further away from the attachment point”. Plantarflex or plantarflexion 288 means movement toward the plantar side 282 of a foot or hand, toward the sole or palm. Dorsiflex or dorsiflexion 290 means movement toward the dorsal side 280 of a foot or hand, toward the top. FIG. 2D is a perspective dorsal view of bones of the foot 200. A transverse plane is the plane that shows the top of the foot. A lateral side 292 means a side furthest away from the midline of a body, or away from a plane of bilateral symmetry of the body. A medial side 294 means a side closest to the midline of a body, or toward a plane of bilateral symmetry of the body. For a Lapidus procedure, the intermetatarsal (IM) angle 296 is the angle to be corrected to remove the hallux valgus (bunion) deformity.

FIG. 2E is a view of a foot illustrating common planes 260 of reference for a human foot. FIG. 2E illustrates a sagittal plane 262 that divides the foot into a right section and a left section half. The sagittal plane 262 is perpendicular to frontal or coronal plane 264 and the transverse plane 266. In the foot, the frontal plane 264 generally runs vertically through the ankle and the transverse plane 266 generally runs horizontally through the midfoot and toes of the foot.

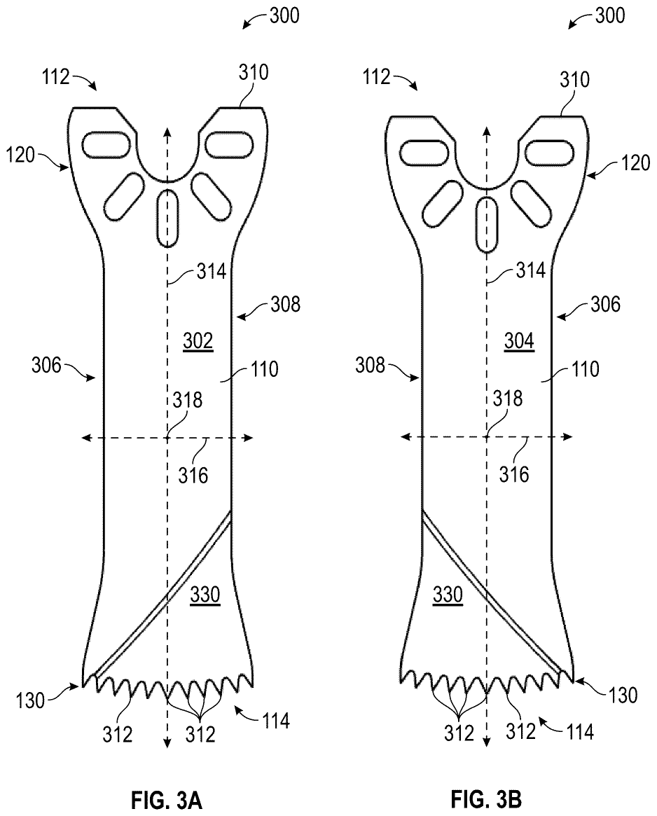

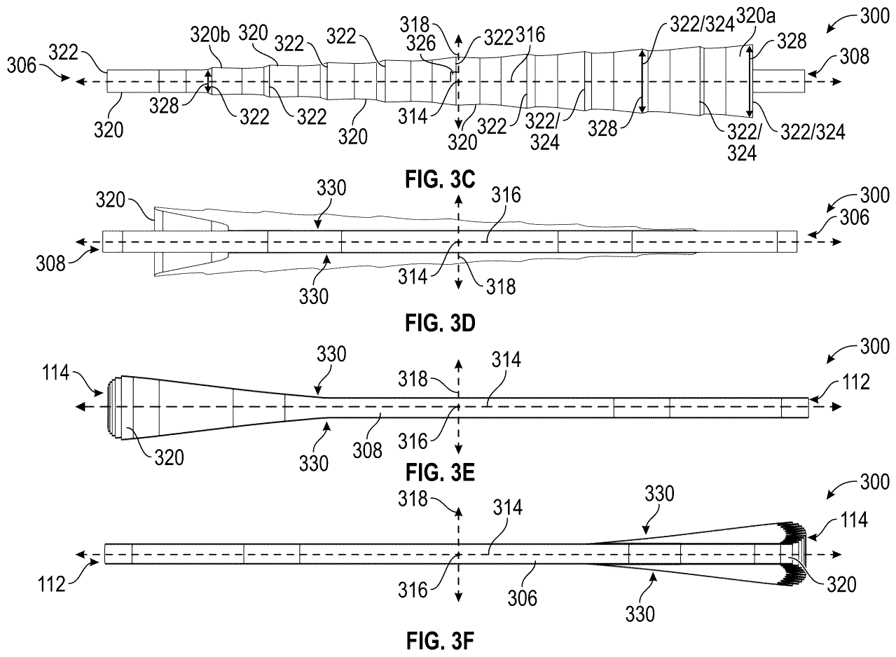

FIG. 3A is a plan view of a superior side and FIG. 3B is a plan view of an inferior side of a wedge osteotomy blade 300 for forming a wedge osteotomy according to one embodiment. The wedge osteotomy blade 300 may have many structures, features, and functions, operations, and configuration similar or identical to those of the apparatus 100 described in relation to FIG. 1, like parts are identified with the same reference numerals. Accordingly, the wedge osteotomy blade 300 may include a body 110, proximal end 112, distal end 114, oscillation mount 120, cutting edge 130, and cutting teeth 132.

In some embodiments, the wedge osteotomy blade 300 includes a body 110 having multiple surfaces and directional axes to define its three-dimensional structure and orientation. The body 110 includes a superior side 302, an inferior side 304, a left side 306, a right side 308, a proximal side 310, and a distal side 312. The proximal side 310 includes the proximal end 112 of the body 110, and the distal side 312 includes the distal end 114 of the body 110.

The body 110 further defines a longitudinal axis 314 that extends between the proximal end 112 and the distal end 114, a lateral axis 316 that extends between the left side 306 and the right side 308, and a vertical axis 318 that extends between the superior side 302 and the inferior side 304. These orthogonal axes may be used as reference frames to define the position, orientation, and geometry of other structural features of the wedge osteotomy blade 300, such as the cutting edge 130, the oscillation mount 120, and/or the cutting teeth 132. The defined axes may also serve as alignment guides for orientation during manufacture, surgical planning, navigation, or intraoperative positioning.

In one embodiment, the distal end 114 of the wedge osteotomy blade 300 includes a plurality of cutting teeth 132. The cutting teeth 132 are positioned along the cutting edge 130 and may be arranged in a linear, arcuate, staggered, and/or irregular pattern. The number, size, spacing, and orientation of the cutting teeth 132 may vary depending on the desired application, such as the size of the wedge resection, the density of the bone, and/or the direction of approach. The cutting teeth 132 may include sharp, serrated, beveled, or textured surfaces, to facilitate bone engagement and material removal.

In some embodiments, at least one cutting tooth 320 includes a bone cutting feature 322. (See FIGS. 3C-3F). The bone cutting feature 322 may be embodied in a variety of designs and configurations. In some embodiments, the bone cutting feature 322 includes a single tip, a single edge, a plurality of tips, a plurality of edges, or the like. These features may be aligned along a common plane, offset from one another, arranged in a staggered or stepped configuration, or otherwise positioned to achieve a desired cutting interaction with bone.

Referring now to FIG. 3C, in one embodiment, the bone cutting feature 322 may be defined, at least in part, by an engagement angle 326 measured relative to the lateral axis 316 and a length 328 measured relative to the vertical axis 318. In another embodiment, the bone cutting feature 322 may include an edge 324 and may be defined, at least in part, by an engagement angle 326 of the edge 324 measured relative to the lateral axis 316 and a length 328 measured relative to the vertical axis 318. In certain embodiments, the engagement angle 326 is 90 degrees. In another embodiment, the engagement angle 326 is in the range of between about 2 degrees and 188 degrees.

In one embodiment, the engagement angle 326 is defined between an edge 324 and the lateral axis 316. In certain embodiments, the engagement angle 326 between the edge 324 and the lateral axis 316 is perpendicular to the lateral axis 316. Those of skill in the art will appreciate that the engagement angle 326 can be any angle between 0 degrees and 180 degrees and can be set to meet particular design objectives. In another embodiment, the engagement angle 326 is measured between an edge 324 and a direction of oscillation of the wedge osteotomy blade 300.

The engagement angle 326 can affect the aggressiveness, directionality, and efficiency of bone cutting. The length 328 can influence the width of the cut, volume of material removed, and/or the slope of a resulting resection. The engagement angle 326 and/or length 328 may be selected to suit particular anatomical structures, resection geometries, or procedural goals include a wedge osteotomy.

In certain configurations, the bone cutting feature 322 of a first cutting tooth 320a positioned closer to the right side 308 of the wedge osteotomy blade 300 is longer than the bone cutting feature 322 of a second cutting tooth 320b positioned closer to the left side 306. Said another way, the length 328 of the first cutting tooth 320a is longer along the vertical axis 318 than the length 328 of the second cutting tooth 320b along the vertical axis 318. Said still another way, the thickness of cutting teeth of a bone cutting feature 322, may progressively increase from one end of a cutting edge, such as the left side 306 of cutting edge 130, to an opposite end of the cutting edge, such as the right side 308 of cutting edge 130.

This variation along the lateral axis 316, for example in length, can create a wedge-shaped or sloped cut profile when the blade is advanced into bone along the longitudinal axis 314. A progressive change in a bone cutting feature 322, such as for example a cutting feature length, across the lateral axis 316 can reduce the need for multiple cutting passes and improve control over angular correction and resection geometry. This design may also enhance visibility and allow the surgeon to better align the blade with anatomical landmarks or correction targets.

FIGS. 3A and 3B illustrate an embodiment of a wedge osteotomy blade 300 that includes at least one relief 330. In the illustrated embodiment, the wedge osteotomy blade 300 includes two reliefs 328 one on the superior side 302 and one on the inferior side 304. In certain embodiments, the relief 330 is positioned between the cutting teeth 132 and the body 110.

The relief 330 may define a recessed, tapered, contoured, or undercut region that provides clearance behind the cutting teeth 132. The relief 330 may serve one or more purposes, including reducing friction between the blade and the bone surface, minimizing contact area to avoid heat buildup, and facilitating smoother advancement or withdrawal of the blade during cutting. In some embodiments, the relief 330 may enhance visibility and tactile feedback for the surgeon, support debris evacuation, or reduce resistance during resection. The geometry, size, and placement of the relief 330 may be selected based on the desired cutting dynamics, blade stiffness, and anatomical application.

FIG. 3D is a proximal side view of wedge osteotomy blade 300 according to one embodiment. FIG. 3D illustrates the space created by the relief 330 on the superior side 302 and the relief 330 on the inferior side 304.

FIG. 3E is a right-side view of wedge osteotomy blade 300 according to one embodiment. FIG. 3E also illustrates the space created by the relief 330 on the superior side 302 and the relief 330 on the inferior side 304.

FIG. 3F is a left-side view of wedge osteotomy blade 300 according to one embodiment. FIG. 3F also illustrates the space created by the relief 330 on the superior side 302 and the relief 330 on the inferior side 304.

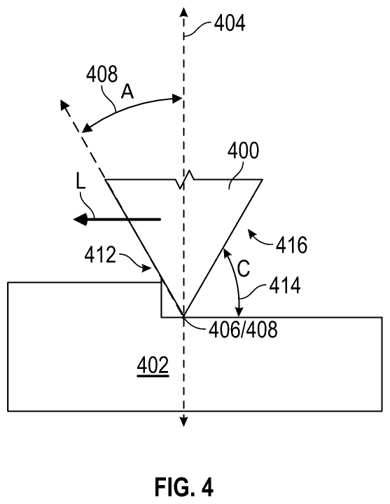

FIG. 4 is a schematic drawing illustrating a rake angle formed between a cutting tool 400 or tooth 400 and a reference plane, showing the tool 400 moving relative to a workpiece 402. FIG. 4 illustrates a cutting operation employing a cutting tool 400 or tooth 400 in engagement with the workpiece 402. The cutting tool 400 is depicted moving horizontally toward the left, as indicated by the directional arrow “L”. A vertical reference axis 404, also referred to as the tool centerline, is illustrated as a dashed line passing through the apex or tip 406 of the cutting tool 400. The cutting edge 408 is the leading edge of the cutting tool 400 that directly engages and removes material from the workpiece 402. The angle labeled as angle “A” is defined as the rake angle 410, which is measured from a reference plane, such as the vertical axis 404 (tool centerline), to a cutting face 412 of the cutting tool 400.

The rake angle 410 influences cutting efficiency, chip formation, and cutting forces experienced during a machining, abrading, or cutting processes. A positive rake angle is defined by the cutting face 412 sloping away from the cutting edge 408 and away from reference axis 404 at an inner side, facilitating efficient cutting by effectively shearing material from the workpiece 402. A positive rake angle is a cutting face 412 that extends to the right of the reference axis 404, in FIG. 4. A negative rake angle is defined by the cutting face 412 sloping away from the tool centerline, aka reference axis 404 at an outer side, providing increased tool strength but may require greater cutting force. A negative rake angle is a cutting face 412 that extends to the left of the reference axis 404, in FIG. 4. The rake angle 410 depicted in FIG. 4 is a negative rake angle.

Regarding the cutting tool geometry, the reference plane (represented here by the vertical axis 404) is typically defined as a plane perpendicular to the cutting direction, “L” and serves as a reference for angle measurements associated with cutting tool geometry. In conjunction with the rake angle 410, other tool angles such as the clearance (relief) angle 414 (represented by angle “C”) and lip angle (not explicitly shown) may also be defined relative to this reference plane (represented here by the vertical axis 404). The clearance angle 414 is formed between the flank or trailing face 416 of the cutting tool 400 and the surface of the workpiece 402 being machined or cut. The clearance angle 414 helps to ensure that only the cutting edge 408 engages the workpiece 402 and reduces rubbing or friction, which can lead to wear and thermal damage. The lip angle is the angle formed between the rake face 412 and the flank or clearance face 414 of the cutting tool 400, and can affect the strength and cutting performance of the cutting tool 400.

Proper selection and combination of rake, clearance, and lip angles contribute to optimizing tool life, cutting performance, and surface finish quality during machining processes. It should be noted that where the cutting tool 400 is an oscillating blade/tool the cutting tool 400 iterates between moving in cutting direction “L” and then moving in a cutting direction opposite direction “L”. Consequently, a cutting face 412 and flank or trailing face 416 in one direction “L” will reverse rolls and/or functions when the cutting tool 400 moves in the cutting direction opposite direction “L”.

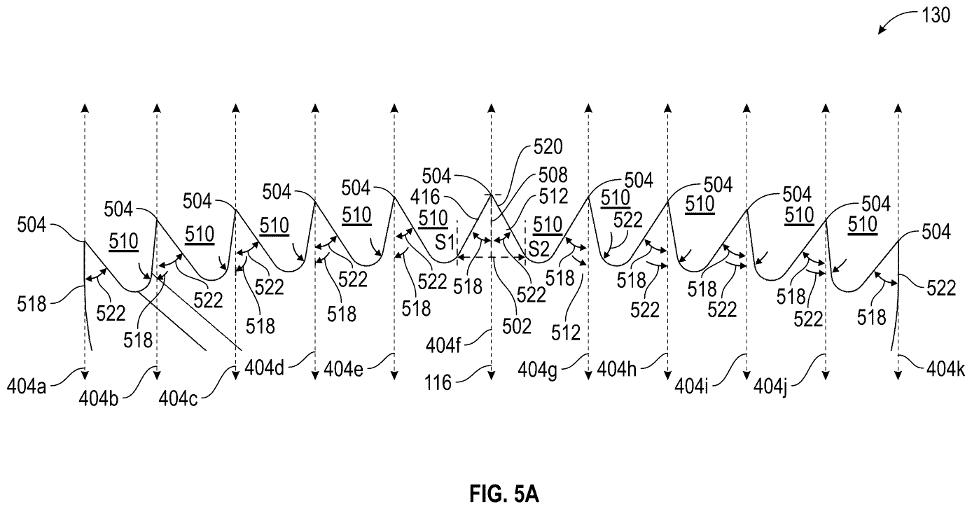

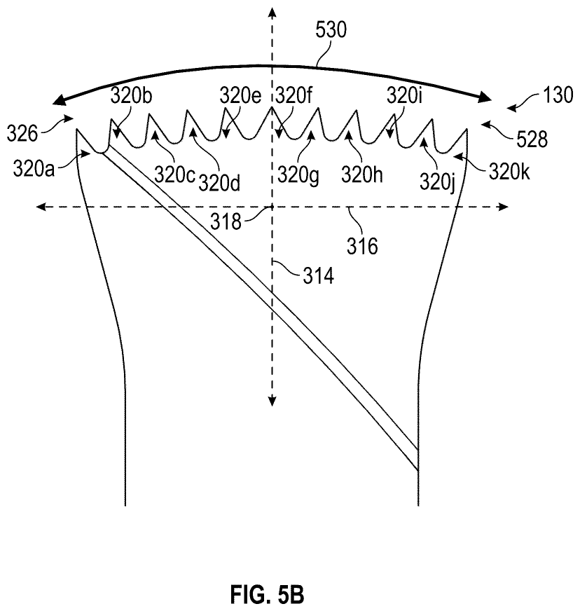

FIG. 5A is closeup view of teeth of wedge osteotomy blade 300 according to one embodiment. The wedge osteotomy blade 300 may have many structures, features, and functions, operations, and configuration similar or identical to those of the wedge osteotomy blade 300 described in relation to FIGS. 1, 3A-3F, like parts are identified with the same reference numerals. Accordingly, the wedge osteotomy blade 300 may include a cutting edge 130, a plurality of cutting teeth 132, with an individual tooth identified by reference numeral 320.

Referring now to FIG. 5A and FIG. 5B, in the illustrated embodiment, the cutting edge 130 includes eleven cutting teeth 132 (each a cutting tooth 320 or tooth 320), in the illustrated embodiment. Each tooth 320 includes a base 502 and a tip 504. The tip 504 serves as a bone cutting feature. The base 502 includes a first side S1 and a second side S2. Each tooth 320 has a width 506 measured from the first side S1 of the base 502 to the second side S2 of the base 502.

A tooth 320 also has a height 508 measured from the base 502 to the tip 504. In one embodiment, the space between teeth 320 may be referred to as a gullet 510. The size of the gullet 510 is one example of and/or representation of a tooth separation distance. A tooth separation distance is a measure of a distance between teeth 320 or a distance between parts of two teeth 320 (full or partial). For partial teeth, the width 506 and/or height 508 may be shortened due to the application and/or due to the position of the partial tooth 320 relative to an edge of the cutting edge 130.

Each tooth 320 can have the same or a different design, shape, size, and/or configuration depending on the application. In certain embodiments, each tooth 320 also includes a superior side 512 and an inferior side (not shown in FIG. 5A). In one embodiment, the superior side 512 is opposite the inferior side. The superior side 512 extends from the base 502 to the tip 504. The inferior side extends from the base 502 to the tip 504. In certain embodiments, the superior side 512 and inferior side are planar. The superior side 512 and inferior side may correspond to the superior side 302 and inferior side 304 of the wedge osteotomy blade 300.

The first side S1 has a first face 516 having a first rake angle 518 and the second side S2 has a second face 520 having a second rake angle 522. FIG. 5A includes a plurality of vertical axes 404a-404k. The vertical axes 404a-404k facilitate identifying the different rake angles for each cutting tooth 320a-320k. In the illustrated embodiment, the cutting edge 130 includes five cutting teeth 320 on one side (cutting teeth 320a-320e) and five cutting teeth 320 on the other side (cutting teeth 320g-320k), in the illustrated embodiment. In this design, the center tooth 320f has a negative first rake angle 518 and a negative second rake angle 522 that are substantially the same. Furthermore, the cutting teeth 320a-320e have first rake angle 518 that progressively increases from about zero degrees to about the same as the second rake angle 522, while the second rake angle 522 is larger and progressively decreases in size. Similarly, the cutting teeth 320g-320k have first rake angle 518 about the same as the second rake angle 522 that progressively increases while the second rake angle 522 progressively decreases in size. In the illustrated embodiment, the rake angles 518,522 for each tooth 320 are negative rake angles.

FIG. 5B is a plan view of a distal end of a wedge osteotomy blade 300 according to one embodiment. The wedge osteotomy blade 300 may have many structures, features, and functions, operations, and configuration similar or identical to those of the wedge osteotomy blade 300 described in relation to FIGS. 1, 3A-3F, like parts are identified with the same reference numerals. Accordingly, the wedge osteotomy blade 300 may include a cutting edge 130, a plurality of cutting teeth 132, with an individual tooth identified by reference numeral 320.

In one embodiment, the cutting edge 130 includes a first side 526 and a second side 528. The width of the cutting edge 130 is measured from the first side 526 to the second side 528. In the illustrated embodiment, the cutting teeth 132 (cutting teeth 320a-320k) are distributed along the cutting edge 130 from the first side 526 to the second side 528.

Advantageously, in certain embodiments, the cutting teeth 132 (cutting teeth 320a-320k) have a nonuniform thickness across the width of the cutting edge 130. As illustrated in FIG. 5B, in one embodiment, the nonuniform thickness is a dimension perpendicular to a height 508 and width 506 of each tooth. In other words, the cutting teeth 132 along the cutting edge 130 have different thicknesses. In an example embodiment, the thickness of cutting tooth 320a is a smallest thickness and the thickness of cutting tooth 320k is the greatest thickness with the thicknesses of cutting teeth between them (cutting teeth 320b-320j) progressively increasing for the cutting teeth moving from the first side 526 to the second side 528 of the cutting edge 130. In alternative embodiments, the variation in thickness of cutting teeth 132 across the cutting edge 130 may follow other patterns or arrangements. For example, the thickness may progressively increase from each side toward the center tooth 320f, decrease progressively from the center toward each side, alternate between thicker and thinner teeth, or otherwise vary to optimize cutting efficiency, blade durability, or debris removal according to specific surgical requirements or cutting applications.

FIG. 5B also illustrates an embodiment in which one of the plurality of teeth comprises a center tooth 320f positioned midway between or approximately midway between first side 526 of the cutting edge 130 and the second side 528 of the cutting edge 130. As described above in relation to FIG. 5A, which is a closeup of the same embodiment, the first rake angle 518 of cutting tooth 320f is about or may be substantially the same as the second rake angle 522. In oscillating cutting operations, it can be advantageous for a center tooth, such as tooth 320f, to contact the bone first, as this initial contact provides a stable reference point and reduces lateral vibration, enhancing the precision and efficiency of the cut. Additionally, having the same rake angle on each cut face of the center tooth 320f provides symmetrical cutting forces, thereby minimizing torque or twisting forces on the blade and reducing uneven wear. Symmetrical rake angles on the center tooth also facilitate consistent chip evacuation from either side, improving the overall cutting efficiency and extending the operational life of the blade.

FIG. 5B illustrates that in the illustrated embodiment, a first plurality of teeth between the first side 526 of the cutting edge 130 and the center tooth 320 f have rake angles 518/522 that bias the tips 504 of the first plurality of teeth away from the center tooth 320f. In the illustrated embodiment, the rake angles 518/522 and/or designs of cutting teeth 320a-320e bias, or direct, the tips 504 away from the center tooth 320f and towards the first side 526. Furthermore, a second plurality of teeth between the second side 528 of the cutting edge 130 and the center tooth 320f have rake angles 518/522 that bias the tips 504 of the second plurality of teeth away from the center tooth. In the illustrated embodiment, the rake angles 518/522 and/or designs of cutting teeth 320g-320k bias, or direct, the tips 504 away from the center tooth 320f and towards the second side 528.

Advantageously, the biasing of the cutting teeth 320a-320e and 320g-320k away from the center tooth 320f provides several benefits. First, the outward biasing assists in effectively evacuating bone chips or debris from the cutting area, reducing clogging or buildup that could otherwise decrease cutting efficiency. Second, this biasing creates a controlled cutting action that helps maintain the blade's stability during operation, decreasing lateral vibration and increasing precision. Furthermore, directing the teeth outward from the central tooth 320f evenly distributes cutting forces across the blade, which reduces stress concentration and helps extend blade life. The symmetrical arrangement of biased teeth on each side of the center tooth also ensures balanced cutting performance during oscillation in either direction, providing consistent results throughout the entire cutting operation.

FIG. 5B illustrates that in certain embodiments the cutting edge 130 defines an arc 530 at a distal end 114 of the wedge osteotomy blade 300. The arc 530 can facilitate use of the wedge osteotomy blade 300 in a wedge osteotomy. For example, in an embodiment in which the cutting teeth 132 towards the first side 526 are thinner than the cutting teeth 132 towards the second side 528, the center tooth 320f, at or near the apex of the arc 530 can have a thickness that is the average of all the cutting teeth 132. As a user directs the operating wedge osteotomy blade 300 towards one or more bones directly along the longitudinal axis 314, the center tooth 320f can contact the bone(s) first. The average thickness of the center tooth 320f can facilitate initial penetration of the cutting edge 130 into the bone(s). As the wedge osteotomy blade 300 continues to move distally along the longitudinal axis 314 more and more cutting teeth 132 engage with bone and begin cutting the bone. Thus, the center tooth 320f and cutting teeth 132 defining an arc 530 can provide a more stable and capable wedge osteotomy blade 300. In this manner, a subset of the cutting teeth 132 engage with bone(s) (i.e., target material) during cutting.

In alternative embodiments, the arc 530 defined by the cutting edge 130 may vary depending on the intended surgical application or cutting requirements. For example, arc 530 can have a more pronounced curvature (i.e., a smaller radius), thereby increasing initial tooth penetration and potentially reducing the force required for entry into bone. Alternatively, the arc 530 can be less pronounced (i.e., a larger radius) to provide a broader initial cutting area, potentially enhancing stability and control during entry into denser or harder bone structures. In certain embodiments, the arc 530 may also be asymmetrical, with the apex positioned closer to one side, thereby accommodating particular surgical approaches or anatomical constraints.

The design of arc 530 advantageously facilitates progressive tooth engagement, meaning fewer teeth engage the target bone initially, reducing overall cutting force requirements and improving initial precision. By engaging a subset of cutting teeth 132 at any one-time during cutting, the design can reduce the load on the blade and provides more predictable and controlled penetration into the target bone. This incremental engagement distributes cutting forces evenly and reduces blade vibration, enhancing precision and operational stability. Furthermore, controlled and progressive engagement of cutting teeth 132 enables effective debris management, facilitating evacuation of bone fragments and reducing potential clogging between adjacent teeth.

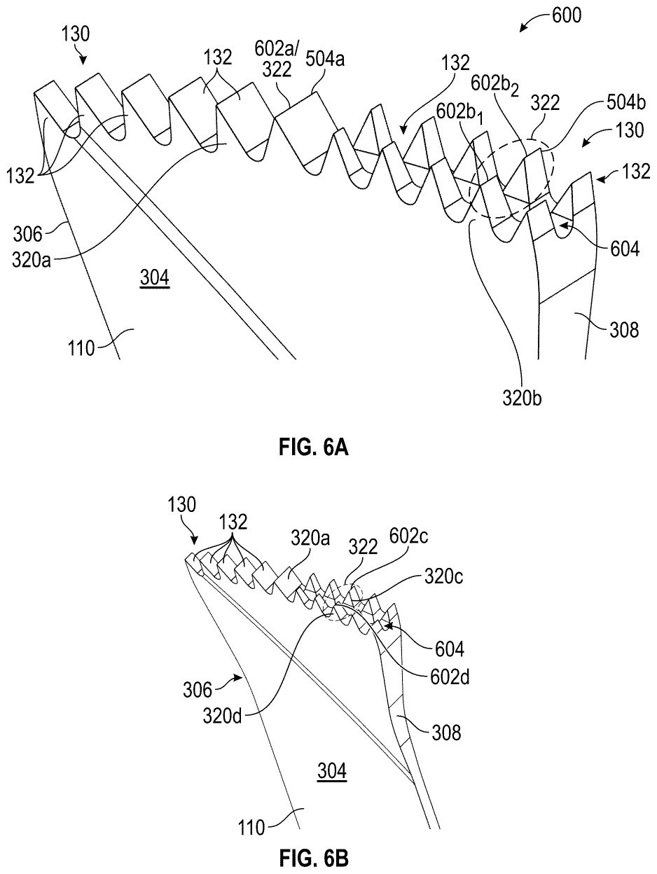

FIG. 6A is closeup view of teeth of a wedge osteotomy blade 600 and FIG. 6B is perspective view of the wedge osteotomy blade 600 according to one embodiment. The wedge osteotomy blade 600 may have many structures, features, and functions, operations, and configuration similar or identical to those of the wedge osteotomy blade 300 described in relation to FIGS. 1, 3A-3F, 5A, 5B like parts are identified with the same reference numerals. Accordingly, the wedge osteotomy blade 600 may include a cutting edge 130, a plurality of cutting teeth 132, with an individual tooth identified by reference numeral 320, and one or more bone cutting features 322.

Those of skill in the art will appreciate that a bone cutting feature 322 can have a variety of forms, shapes, designs, configurations, and/or embodiments. The wedge osteotomy blade 600 illustrates a few examples. In one embodiment, a bone cutting feature 322 is embodied as a single edge 602a at a tip 504a of a cutting tooth 320, such as cutting tooth 320a.

Alternatively, or in addition, the bone cutting feature 322 can be embodied as at least two edges 602b1, 602b2 of a cutting tooth 320b. In such an embodiment, the cutting tooth 320b may include a channel 604 that extends through the tip 504b. In one embodiment, the channel 604 may extend from the second side 528 of the cutting edge 130 towards the first side 526. In the illustrated embodiment, the channel 604 may pass through the tips 504 and divide a single cutting tooth 320 into two aligned teeth. In this design, in the illustrated embodiment, the bone cutting feature 322 may include two edges 602b1, 602b2 of a single cutting tooth 320b.

Referring now to FIG. 6B, of course, a bone cutting feature 322 can also be implemented by way of an edge 602c, 602d of at least two cutting teeth 320c, 320d which are separated by a channel 604. The edges 602c, 602d and/or cutting teeth 320c, 320d may be aligned along the vertical axis 318 of the wedge osteotomy blade 600 or not depending on the desired application and/or function for a particular cutting edge 130. In certain embodiments, the channel 604 can be configured to encourage evacuation of bone debris during cutting.

Those of skill in the art will appreciate that the bone cutting feature 322 includes one or more structures that contact and/or engage with a target material (i.e., bone) during cutting. In certain embodiments, the bone cutting feature 322 may be implemented as one or more edges 602 of one or more cutting teeth 132. Of course, other structures can be used instead of or together with edges 602 for a bone cutting feature 322.

Referring still to FIGS. 6A and 6B, a cutting edge 130 can have a variety of configurations. In the illustrated embodiment, the cutting edge 130 can include a single row of cutting teeth 132 on a first side (i.e., the left side 306) and at least two rows of cutting teeth 132 on a second side (i.e., right side 308). The illustrated embodiment, also includes at least one channel 604 that intersects cutting teeth 132 of the cutting edge 130. Those of skill in the art will appreciate that the channel 604 can be start and/or end at any point along the cutting edge 130 and can include one or more of the cutting teeth 132.

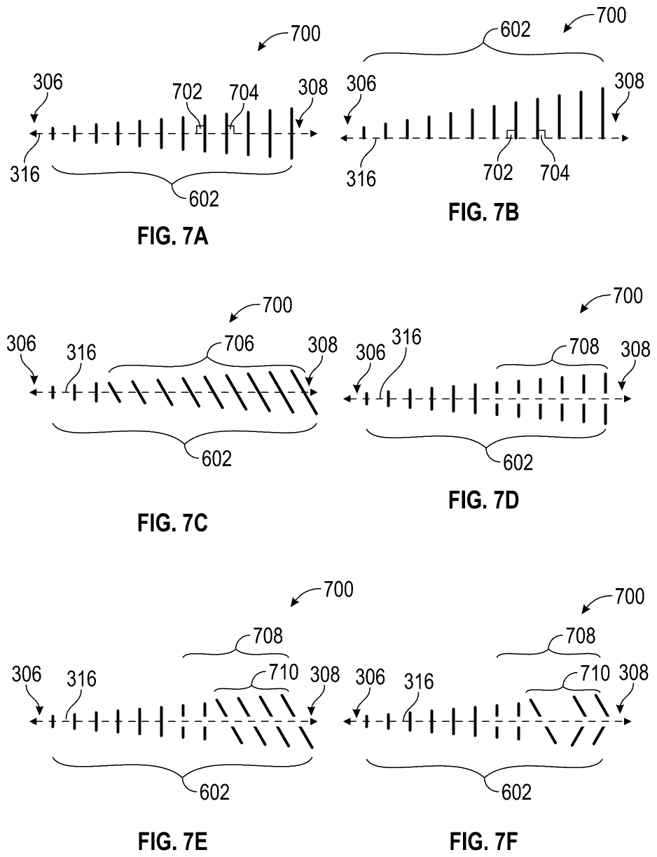

FIGS. 7A-7F illustrate different patterns 700 for bone cutting features 322 according to one embodiment. As explained above, a variety of designs and/or configurations can be used for a bone cutting feature 322. In certain embodiments, the bone cutting feature 322 is implemented using one or more edges 602 of one or more cutting teeth 132. FIGS. 7A-7F illustrate different patterns for how these edges 602 can be oriented with respect to the target material. Thus, the patterns shown illustrate the position and/or orientation of the edges 602 from the perspective of the bone. Said another way, the patterns illustrate the position and/or orientation of the edges 602 relative to a lateral axis 316.

FIG. 7A illustrates a plurality of edges 602 of a cutting edge 130 according to one pattern 700. In this embodiment, the tip 504 may be centered along the lateral axis 316 (along the vertical axis 318 of the wedge osteotomy blade 300). In this configuration, the edges 602 extend the same distance away from the superior side 302 of the wedge osteotomy blade 300 as from the inferior side 304. This can be advantageous such that a user is assured that alignment of the lateral axis 316 with a desired trajectory will cause an even resection on each side of the lateral axis 316.

In the pattern 700 of FIG. 7B, rather than centering the edges 602 along the lateral axis 316 a common end of the edges 602 is aligned with the lateral axis 316. Of course, the opposite end of the edges 602 can be aligned with the lateral axis 316. Alternatively, or in addition, alternating ends of adjacent edges 602 from the left side 306 to the left side right side 308 can be aligned with the lateral axis 316.

FIGS. 7A-7F and the different patterns 700 for bone cutting features 322 that are embodied using one or more edges 602 also illustrate an engagement angle for each edge 602, according to certain embodiments. The bone cutting feature 322 can include a first engagement angle 702 and a second engagement angle 704. An engagement angle is an angle measured between the edge 602 and the lateral axis 316. In certain embodiments, each edge 602 has both a first engagement angle 702 and a second engagement angle 704 and the first engagement angle 702 and the second engagement angle 704 together sum to 180 degrees. Those of skill in the art will appreciate that the engagement angles can be varied and/or adjusted depending on the application. In certain embodiments, the engagement angles can be varied to facilitate cutting precision and/or debris evacuation.

In FIGS. 7A and 7B, the first engagement angle 702 and second engagement angle 704 are each 90 degrees. In such embodiments, two or more cutting teeth 132 of a plurality of cutting teeth 132 can include a bone cutting feature 322 and the engagement angles 702,704 of each or of all of the two or more bone cutting features 322 can be substantially the same. In another embodiment, two or more cutting teeth 132 of the plurality of cutting teeth 132 include a bone cutting feature 322 and the engagement angles (702 and/or 704) of the two or more bone cutting features 322 are different.

Of course, in various embodiments, the same engagement angles 702 for two or more edges 602 can be the same and in other embodiments the same engagement angles 702 for the two or more edges 602 can be different. Similarly, the same engagement angles 704 for two or more edges 602 can be the same and in other embodiments the same engagement angles 704 for the two or more edges 602 can be different. Thus, embodiments of the edges 602 of the cutting teeth 132 and/or first engagement angles 702 and/or second engagement angles 704 can be implemented with any permutation of the same or different first engagement angles 702 and/or second engagement angles 704.

FIG. 7C illustrates a pattern 700 for an alternative embodiment of a wedge osteotomy blade 300 in which a subset 706 of bone cutting feature 322 such as edges 602 have a first engagement angle 702 and a second engagement angle 704 that are not 90 degrees. In the illustrated embodiment, the first engagement angle 702 may be about 45 degrees and the second engagement angle 704 may be about 135 degrees.

FIG. 7D illustrates a pattern 700 for an alternative embodiment of a wedge osteotomy blade 600 in which a subset 708 of bone cutting feature 322 implemented as edges 602 are divided, for example by a channel 604. Alternatively, or in addition, the bone cutting feature 322 of the subset 708 may be implemented by two or more rows of cutting teeth 132 that are aligned to produce the pattern 700 of FIG. 7D. In the illustrated embodiment, each of the first engagement angles 702 and a second engagement angles 704 are 90 degrees.

FIG. 7E illustrates a pattern 700 for an alternative embodiment of a wedge osteotomy blade in which a subset 708 of bone cutting feature 322 implemented as edges 602 are divided, for example by a channel 604. In addition, a subset 710 of subset 708 may be implemented by two or more rows of cutting teeth 132 with edges 602 that have first engagement angles 702 and a second engagement angles 704 are not 90 degrees. Other edges of the subset 708 have first engagement angles 702 and a second engagement angles 704 are 90 degrees.

FIG. 7F illustrates a pattern 700 for an alternative embodiment of a wedge osteotomy blade in which a subset 708 of bone cutting feature 322 implemented as edges 602 are divided or formed as separate edges 602. In addition, a subset 710 of subset 708 may be implemented by two or more rows of cutting teeth 132 with edges 602 that have first engagement angles 702 and a second engagement angles 704 are not 90 degrees. Other edges 602 of the subset 708 have first engagement angles 702 and a second engagement angles 704 are 90 degrees. Certain edges 602 of the subset 710 may include first engagement angles 702 that are acute and second engagement angles 704 that are obtuse.

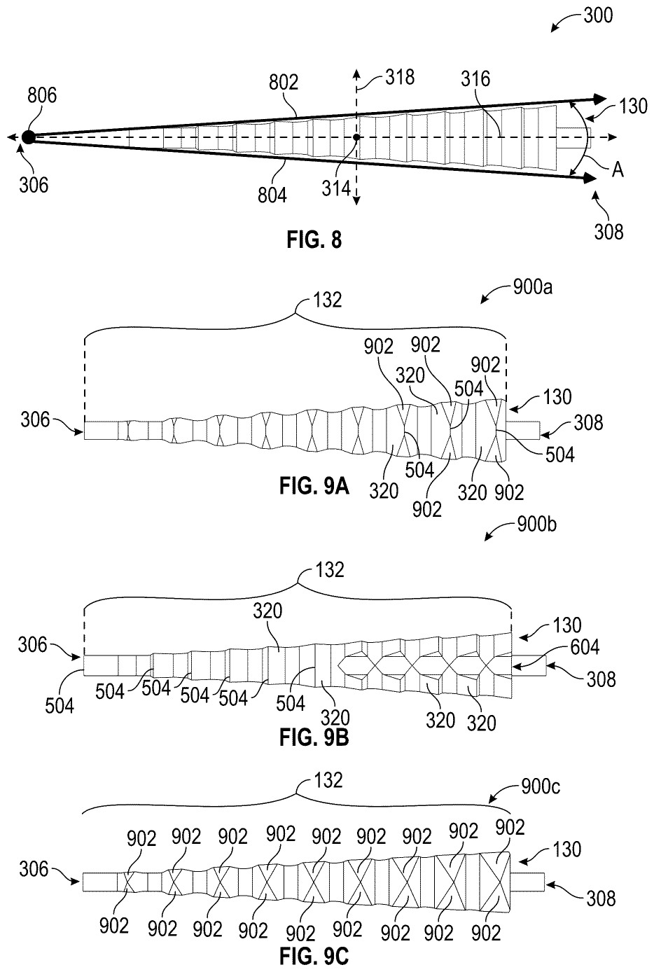

FIG. 8 is a distal side view of wedge osteotomy blade 300 according to one embodiment. The wedge osteotomy blade 300 includes a cutting edge 130 and a plurality of cutting teeth 132, with an individual tooth identified by reference numeral 320. The longitudinal axis 314, lateral axis 316, and vertical axis 318 are also illustrated relative to the cutting teeth 132 and cutting edge 130.

In the illustrated embodiment, the one or more of the plurality of cutting teeth 132 define a superior cut line 802 and one or more of the plurality of cutting teeth 132 define an inferior cut line 804. In certain embodiments, the superior cut line 802 is a line or trajectory identifying where the wedge osteotomy blade 300 will cut in the bone when used. In one embodiment, the superior cut line 802 is on the same side as the superior side 302 of the wedge osteotomy blade 300. Similarly, the inferior cut line 804 is a line or trajectory identifying where the wedge osteotomy blade 300 will cut in the bone when used. In one embodiment, the inferior cut line 804 is on the same side as the inferior side 304 of the wedge osteotomy blade 300.

In one embodiment, the superior cut line 802 and inferior cut line 804 converge at a vertex 806. The vertex 806 defines and/or has a wedge angle A. The wedge angle A identifies the angle for a bone wedge that the wedge osteotomy blade 300 is configured to form in one or more bones during an osteotomy. In certain embodiments, the vertex 806 corresponds to an apex for a bone wedge a surgeon desires to resect from one or more bones. Advantageously, a surgeon can position the vertex 806 next to a distal surface of a bone. That surface can be cortical bone. The cortical bone may be preserved during a resection using the wedge osteotomy blade 300 so that the remaining cortical bone serves as a living hinge and/or pivot point for the wedge osteotomy. Some surgeons may deploy a K-wire and then position the vertex 806 at and more the vertex 806 along the K-wire to facilitate guiding the wedge osteotomy blade 300 for a resection.

Those of skill in the art will appreciate that the wedge angle A can be almost any acute angle. In one embodiment, the wedge angle A can range from between about 3 degrees to about 10 degrees. In one embodiment, the wedge angle A is, or is about, 6 degrees.

FIG. 8 also illustrates that in one embodiment, the cutting edge 130 can include a nonuniform thickness. As but one example, cutting teeth 132 on and towards the left side 306 are thinner along the vertical axis 318 than cutting teeth 132 on and towards the right side 308. This change in thickness can be progressive and can be implemented by the thickness of each cutting tooth 320.

Alternatively, or in addition, a wedge-shaped osteotomy can be performed using a wedge osteotomy blade 300 by varying the length of the bone cutting feature 322 (i.e., edges 602) along the width of the cutting edge 130. In particular, cutting teeth 132 on and towards the left side 306 can have a shorter edge length along the vertical axis 318 than cutting teeth 132 on and towards the right side 308.

FIGS. 9A, 9B, and 9C are distal side views of an alternative embodiments of a wedge osteotomy blade 900a, 900b, 900c. The wedge osteotomy blades 900a, 900b, 900c may have many structures, features, and functions, operations, and configuration similar or identical to those of the wedge osteotomy blade 300 described in relation to FIGS. 1, 3A-3F, like parts are identified with the same reference numerals. Accordingly, the wedge osteotomy blades 900a, 900b, 900c may include a cutting edge 130, a plurality of cutting teeth 132, with an individual tooth identified by reference numeral 320, a left side 306, and a right side 308.

Wedge osteotomy blades 900a, 900b, 900c illustrate a few of the many alternative designs for the cutting edges 130 and/or for the cutting teeth 132. Wedge osteotomy blade 900a includes a cutting teeth 132. Each cutting tooth 320 includes one or more bevels 902. The bevels can extend from a side of the cutting tooth 320 to the tip 504. Furthermore, the bevels 902 can extend from an origin side to the tip 504 at any acute angle. In FIG. 9A the bevels 902 include two bevels 902 on each cutting tooth 320.

In FIG. 9B, the cutting teeth 132 do not include bevels 902, however cutting teeth 132 toward the right side 308 have a channel 604 that pass through some of their tips 504. In FIG. 9C, the cutting teeth 132 do include bevels 902. These bevels 902 are configured to meet at the tip 504 such that the tip 504 comprises a point rather than an edge.

The wedge osteotomy blades 900a, 900b, and 900c shown in FIGS. 9A-9C illustrate some of the possible variations for designing cutting edges and cutting teeth configurations. It should be understood that other embodiments having different shapes, numbers, arrangements, orientations, bevel configurations, channels, or combinations thereof, are also possible. Thus, the designs described herein are exemplary and not limiting; additional variations and modifications that achieve similar functionality and objectives fall within the scope of the present disclosure.

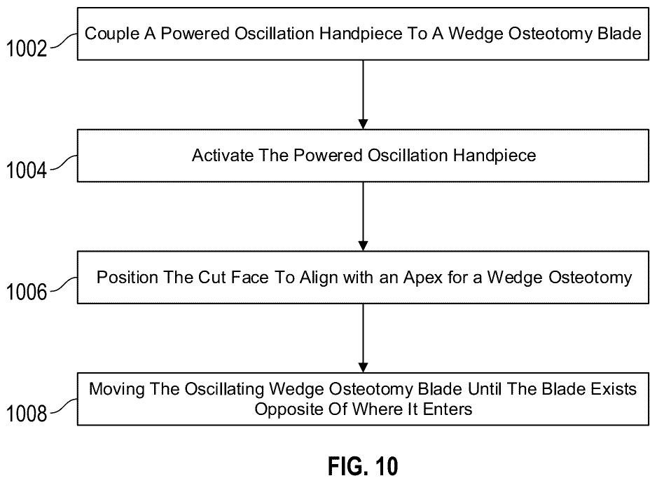

FIG. 10 is a flowchart diagram depicting a method 1000 or process 1000 for performing a wedge osteotomy, according to one embodiment. In some implementations, one or more process blocks of FIG. 10 may be performed in whole or in part by a surgeon, an apparatus, device and/or system of the present disclosure. As shown in FIG. 10, process 1000 may include coupling a powered oscillation handpiece to a wedge osteotomy blade (block 1002). The wedge osteotomy blade may include a body having a proximal end, a distal end. The wedge osteotomy blade may further include an oscillation mount at the proximal end. The oscillation mount may be configured to couple the wedge osteotomy blade to a powered oscillation handpiece. The wedge osteotomy blade may include a cutting edge at the distal end of the body. The cutting edge may include a plurality of cutting teeth that define a cut face.

Advantageously, a thickness of the cut face defined by the plurality of cutting teeth increases laterally across the cutting edge. In another embodiment, the plurality of cutting teeth have a nonuniform thickness across a width of the cutting edge (from one side to the opposite side). In certain embodiments, the cutting edge includes a plurality of cutting teeth 132 that define a cut face for the wedge osteotomy blade. In another embodiment, a thickness of the plurality of cutting teeth varies across the cutting edge. In another embodiment, teeth thickness increases laterally across the cutting edge. In another embodiment, the plurality of cutting teeth have a thickness that increases from a first lateral side to a second lateral side across the width of the cutting edge.

As further shown in FIG. 10, process 1000 may include activating the powered oscillation handpiece (block 1004). Next, process 1000 may include positioning the cut face of the wedge osteotomy blade such that a thinnest part of the cut face substantially aligns with an apex for a wedge osteotomy (block 1006). The wedge osteotomy includes two planes that connect to the apex and extend to at least one bone surface opposite the apex. In one embodiment, the at least one bone surface includes cortical bone of one or more bones.

Next, process 1000 may include moving the oscillating wedge osteotomy blade into the one or more bones until the oscillating wedge osteotomy blade exits the one or more bones on a side of the one or more bones opposite of where the oscillating wedge osteotomy blade enters the one or more bones (block 1008). Some in the medical industry may refer to such movement of the oscillating wedge osteotomy blade into the one or more bones as a plunge motion or plunge cut.

In certain embodiments, the process 1000 may also include a cut face having at least one thicker region and at least one thinner region. Further, the process 1000 may include that moving the oscillating wedge osteotomy blade into the one or more bones forms the wedge osteotomy in a single pass. In this single pass a progressively increasing thickness of the resection can result from the thicker region creating a greater separation between the two planes of the wedge osteotomy and the thinner region creating a smaller separation between the two planes of the wedge osteotomy.

Although FIG. 10 shows example blocks of process 1000, in some implementations, process 1000 may include additional blocks, fewer blocks, different blocks, or differently arranged blocks than those depicted in FIG. 10. Additionally, or alternatively, two or more of the blocks of process 1000 may be performed in parallel.

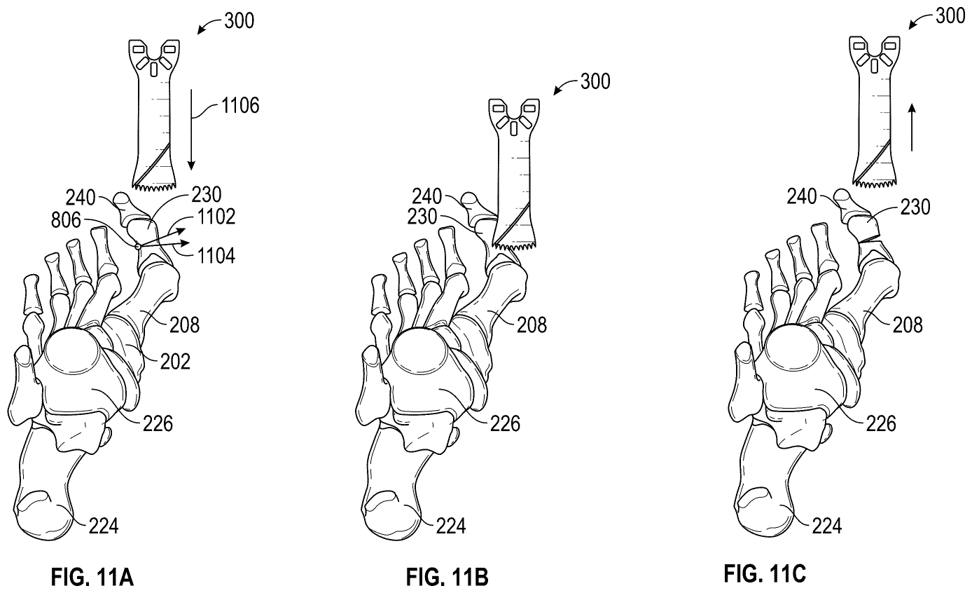

FIGS. 11A, 11B, and 11C illustrate stages in a surgical procedure using a wedge osteotomy blade according to one embodiment. Advantageously, the surgical procedure using the wedge osteotomy blade enables a surgeon to resect a bone wedge in a single pass. In certain embodiments, a surgeon may resect an initial bone wedge having a smaller wedge angle (i.e., 1 or 2 degrees) and then subsequently resect using the same approach and positioning and alignment and but with a wedge osteotomy blade having a larger wedge angle (i.e., 6 degrees). In this manner, each resection may be done in a single pass however, the first may be an initial resection to trial a particular correction angle amount or review an initial correction amount and the subsequent resection may be to reach a final or desired correction angle.

FIG. 11A illustrates the bones of a foot 200. Many bones of the foot 200 are illustrated. A few are numbered for reference such as the calcaneus 224, tibia 226, medial cuneiform 202, first metatarsal 208, first proximal phalange 230, and first distal phalange 240. While the wedge osteotomy blade 300 may be used with a variety of surgical procedures, FIGS. 11A, 11B, 11C illustrate an Akin osteotomy. For this surgical procedure, a surgeon desires to make a resection of the first proximal phalange 230 and form a bone wedge, a wedge osteotomy. In certain embodiments, a surgeon has coupled the wedge osteotomy blade 300 to a powered oscillation handpiece and activated the powered oscillation handpiece.

FIG. 11A illustrates a stage of the resection, according to one embodiment. A surgeon has identified where the desired vertex 806 for the resection is to be (the apex for the bone wedge). The desired planes for the bone wedge are illustrated by arrows 1102 and 1104. FIG. A also illustrates that in one embodiment, a surgeon performs the resection by approaching the first proximal phalange 230 from the dorsal side. The surgeon desires to make a plunge cut from the dorsal side of the first proximal phalange 230 to the plantar side as indicated by arrow 1106.

FIG. 11B illustrates a stage in which the wedge osteotomy blade 300 on the surface and/or initiating the resection of the first proximal phalange 230. Using the wedge osteotomy blade 300 and this approach the apex of the bone wedge will be at or near a lateral cortex of the first proximal phalange 230 and the resulting bone wedge will open on the medial side of the first proximal phalange 230. Next, a surgeon will advance the wedge osteotomy blade 300 until the wedge osteotomy blade 300 exits the first proximal phalange 230 on the plantar side.

FIG. 11C illustrates a stage of the surgical procedure after one single pass with the wedge osteotomy blade 300 and the wedge osteotomy blade 300 has been retracted from the bone opening. Advantageously, the wedge osteotomy blade 300 has formed a bone wedge with planes of the wedge defining a desired wedge angle matching the wedge angle defined for the wedge osteotomy blade 300 selected for the resection. A surgeon cannot proceed with an Akin osteotomy and close the bone wedge or perform any other steps of the surgical procedure, as desired.

The wedge osteotomy blade described herein provides several advantages for performing precise and controlled wedge osteotomies. By utilizing a single-pass approach, the blade facilitates rapid and accurate formation of bone wedges, minimizing intraoperative time and reducing procedural complexity. Additionally, the predefined wedge angle integrated into the blade design enables consistent, reproducible resections, thereby enhancing surgical precision and predictability of outcomes. The controlled plunge-cut method described helps preserve bone integrity and surrounding soft tissues, potentially decreasing surgical trauma and improving recovery times. While these represent several potential advantages, additional benefits and efficiencies may become apparent in clinical practice based upon specific procedural contexts and individual surgeon techniques.

Embodiments of wedge osteotomy blades described herein offer several clinical advantages. First, by incorporating nonuniform tooth geometry, variable tooth thickness, and progressive cutting-edge profiles, the blades facilitate controlled wedge formation in a single surgical pass. This approach reduces operative time significantly compared to traditional multi-pass techniques, potentially decreasing patient exposure to anesthesia, surgical risk, and recovery duration. The blades' design features, including progressively increasing tooth thickness and length across the cutting edge, allow surgeons to precisely shape bone wedges that closely match predetermined correction angles, thus enhancing the predictability and consistency of surgical outcomes.

Moreover, the asymmetrical and varied tooth profiles/designs and rake angles detailed in various embodiments enhance cutting performance by effectively distributing cutting forces across the blade during operation. Such balanced force distribution reduces lateral vibration and blade chatter, promoting smoother, more controlled bone cutting. The controlled and deliberate geometry of the teeth, including nonuniform spacing, size variations, and strategically positioned relief areas, improve debris evacuation from the cutting zone. Improved debris management enhances the surgical visibility and accuracy, minimizing the risk of unintended bone trauma or incomplete resection, further contributing to successful surgical outcomes.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

1. An apparatus for forming a wedge osteotomy, the apparatus comprising:
a body having a proximal end, a distal end;
an oscillation mount at the proximal end, the oscillation mount configured to couple the apparatus to a powered oscillation handpiece;
a cutting edge at the distal end of the body, the cutting edge comprising a plurality of cutting teeth; and
wherein the plurality of cutting teeth have a nonuniform thickness across the width of the cutting edge.

2. The apparatus of claim 1, wherein the nonuniform thickness is a dimension perpendicular to a height and a width of each tooth.

3. The apparatus of claim 1, wherein the thickness of the plurality of cutting teeth progressively increases from one end of the cutting edge to an opposite end of the cutting edge.

4. The apparatus of claim 2, wherein each tooth of the plurality of cutting teeth comprises:
a base having a first side and a second side;
a tip;
a height measured from the base to the tip;
a width measured form the first side of the base to the second side of the base;
a superior side that extends from the base to the tip;
an inferior side that extends from the base to the tip; and
wherein the first side has a first face having a first rake angle and the second side has a second face having a second rake angle.

5. The apparatus of claim 4, one of the plurality of teeth comprises a center tooth positioned midway between a first side of the cutting edge and a second side of the cutting edge, wherein the first rake angle is substantially the same as the second rake angle.

6. The apparatus of claim 5, wherein:
a first plurality of teeth between the first side of the cutting edge and the center tooth have rake angles that bias the tips of the first plurality of teeth away from the center tooth; and
a second plurality of teeth between the second side of the cutting edge and the center tooth have rake angles that bias the tips of the second plurality of teeth away from the center tooth.

7. The apparatus of claim 1, wherein the cutting edge comprises a single row of cutting teeth on a first side and at least two rows of teeth on a second side.

8. The apparatus of claim 1, wherein the cutting edge comprises at least one channel that intersects cutting teeth of the cutting edge and wherein the cutting edge defines an arc at the distal end of the apparatus such that a subset of the plurality of cutting teeth engage with a target material during cutting.

9. A wedge osteotomy blade for forming a wedge osteotomy, the wedge osteotomy blade comprising:
a body having a proximal end, a distal end, a superior side, an inferior side, a left side, a right side, a proximal side that includes the proximal end, a distal side that includes the distal end, and a longitudinal axis that extends between the proximal end and the distal end, a lateral axis that extends between the left side and the right side, and a vertical axis that extends between the superior side and the inferior side;
an oscillation mount at the proximal end, the oscillation mount configured to couple the wedge osteotomy blade to a powered oscillation handpiece;
a plurality of cutting teeth at the distal end of the body;
wherein at least one cutting tooth comprises a bone cutting feature, the bone cutting feature comprising an engagement angle measured relative to the lateral axis and a length measured relative to the vertical axis; and
wherein the bone cutting feature of a first cutting tooth is longer than the bone cutting feature of a second cutting tooth, the first cutting tooth positioned closer to the right side than the second cutting tooth.

10. The wedge osteotomy blade of claim 9, wherein the bone cutting feature comprises an edge, and the engagement angle is perpendicular to the lateral axis.

11. The wedge osteotomy blade of claim 9, wherein the bone cutting feature comprises at least two edges of a single cutting tooth, the single cutting tooth comprising a channel in the tip of the single cutting tooth.

12. The wedge osteotomy blade of claim 9, wherein the bone cutting feature comprises an edge of two separate cutting teeth, the two separate cutting teeth separated by a channel.

13. The wedge osteotomy blade of claim 9, wherein the bone cutting feature comprises a first engagement angle and a second engagement angle, the first and second engagement angles summing to 180 degrees.

14. The wedge osteotomy blade of claim 9, wherein two or more cutting teeth of the plurality of cutting teeth comprise a bone cutting feature and the engagement angles of the two or more bone cutting features are substantially the same.

15. The wedge osteotomy blade of claim 9, wherein two or more cutting teeth of the plurality of cutting teeth comprise a bone cutting feature and the engagement angles of the two or more bone cutting features are different.

16. The wedge osteotomy blade of claim 9, wherein:
one or more of the plurality of cutting teeth define a superior cut line;
one or more of the plurality of cutting teeth define an inferior cut line; and
wherein the superior cut line and the inferior cut line converge at a vertex having a wedge angle.

17. The wedge osteotomy blade of claim 16, wherein the wedge angle ranges from between about 3 degrees to about 10 degrees.

18. The wedge osteotomy blade of claim 9, further comprising at least one relief between the plurality of cutting teeth and the body.

19. A method for performing a wedge osteotomy, the method comprising:
coupling a powered oscillation handpiece to a wedge osteotomy blade, the wedge osteotomy blade comprising:
a body having a proximal end, a distal end;
an oscillation mount at the proximal end, the oscillation mount configured to couple the wedge osteotomy blade to the powered oscillation handpiece;
a cutting edge at the distal end of the body, the cutting edge comprising a plurality of cutting teeth that define a cut face; and
wherein a thickness of the cut face defined by the plurality of cutting teeth increases laterally across the cutting edge;
activating the powered oscillation handpiece such that the wedge osteotomy blade oscillates;
positioning the cut face such that a thinnest part of the cut face substantially aligns with an apex for a wedge osteotomy, the wedge osteotomy comprising two planes that connect to the apex and extend to at least one bone surface opposite the apex, the at least one bone surface comprising cortical bone of one or more bones; and
moving the oscillating wedge osteotomy blade into the one or more bones until the oscillating wedge osteotomy blade exits the one or more bones on a side of the one or more bones opposite of where the oscillating wedge osteotomy blade enters the one or more bones.

20. The method of claim 19, wherein the cut face comprises at least one thicker region and at least one thinner region and wherein moving the oscillating wedge osteotomy blade into the one or more bones forms the wedge osteotomy in a single pass, wherein the progressively increasing thickness of the resection results from the thicker region creating a greater separation between the two planes of the wedge osteotomy, and the thinner region creating a smaller separation between the two planes of the wedge osteotomy.
